SEQUENCE LISTING (19 sequences)

SEQ ID NO: 1 - protein, 189 aa, Homo sapiens
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRAKLEEQAQQIRLQAEAFQARLKSWFEPLVEDMQRQWAGLVEKVQAAVGTSAAPVPSDNH

SEQ ID NO: 2 - DNA, 567 bp, Homo sapiens
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcac

SEQ ID NO: 3 - DNA, 512 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttc

SEQ ID NO: 4 - DNA, 1147 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttctgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 5 - DNA, 386 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggctt

SEQ ID NO: 6 - DNA, 2355 bp, Artificial Sequence
  Description of Artificial Sequence Synthetic polynucleotide
agactctgtctcaaaaaaaaaaaaaaaaaaaaaaaaagagagagagaaaaaggaagttagaaaaacagccctagaggccctacattctgagtaataggagttccagaaaggaagtgattgctgcacaacataaatttgaaaagaaagagaagtgagaaaatagagggaaggaaatcaaagaaataatccaacttctgaaaagtaaagaatgagcttccagcgggaaagtgcctgttgagtgcaggcacagtggaggaaatgaagctgggtgtgttgccaggagttggaaacttgtctgagcaacatagcgcgaccctgtgtctacaaaaaaataaaaacaaaacaaaaaacaaccaaagacttccgaaacagaatggctttagcctgctcaaccgcacactggcacctggccaacagcatctcttcatgattctgaaggacaacgatctgcagctcagccaagcatcagccatctatggcctaggatgcaagaattcagcaatgttaccttcgagtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 7 - DNA, 1021 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggctttgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 8 - DNA, 2229 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggcttgagtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 9 - protein, 62 aa, Homo sapiens
MDPNCSCAAAGVSCTCASSCKCKECKCTSCKKSCCSCCPVGCAKCAQGCICKGASEKCSCCA

SEQ ID NO: 10 - DNA, 420 bp, Homo sapiens
actccgccttccacgtgcacccactgcctcttcccttctcgcttgggaactctagtctcgcctcgggttgcaatggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccagggctgcatctgcaaaggggcatcggagaagtgcagctgctgcgcctgatgtcgggacagccctgctcccaagtacaaatagagtgacccgtaaaatccaggattttttgttttttgctacaatcttgacccctttgctacattcctttttttctgtgaaatatgtgaataataattaaacacttagacttgaaaaaaaaaaaaaaaaaaa

SEQ ID NO: 11 - DNA, 2025 bp, Homo sapiens
gcaacctccaccttccggtttcaagcgattctcctgcctcagcctcccaagtagttgggattacaggcacccgccaccacgcctggctaatttttatatttttagtaaagacagggtttcaccattttggccaggctggtcttgaacccctgatctcgtgatccacccacgttggcctcccaaagtgctggaattacaggcgtgagccaccatgcctggcccacattacattcttactcacctccccctaccatggaattttattccacagatatgctattggtttagctactatatgtatatctgtgttttatacataaagcacaagaaccttccagaaccaattttcgccaccttggaagtaataccacctctactaagaatgcacagcatagaccataaaacctcaatgctaagttcaaatattggccctaccacacatgagctgtgtggtcttgtacaagttacataacttctcctccttgtctcaaactcctcacatataagatgaggataataatagtacctgcggccacacacagtggcttaaacgtgtaatcccagcactttgggaggctgcggcagaaggatcactcaaactcaggagttcaagagcagcctgggtacatggcgaaactctgtctctacaaaaaatacaaaaattagctgggtgtggtgatgtgtgcctgtagttccagctacttgggaggctgaggtgagaggatcgttcgagcccaggagatcaaggatgcagtgagctatgatcatgtggctgcactccagcctggataacagagccagaccctgtctgaaagaaacaaaaacaaaaacattagcacctgcatcatagggtcactgggggcactacatgagttcatgtacatcgaggacttaggacattgcctcaggcagacctagtgctgcacaattgcttatgtaattattcccaaatttctccagggcccacagaagaacatggaagtatcttggtttggcaattaaggtgaatcacattctcactctccttttctgcatctctaccccacattcccacaaagctttattcacaccaagtctccagtccttgcctgcattgtgtgatgggtgcctgcagtgatgggtggggacacccatcactgtccagggcgtccccaccatcctcacagcctctctgtctggcctcctgcctttgagccagcccaccacactctcatttctctgcccagcagaaaccaaactgtcctctgcatttactgtctcaactggaagagaaatgcagaatgacaaagaacttgtgaacaagggtcagctccaacagagagtgaagccaaaggggctgggcagaaagagagatgaagacgggggtctgaggaataaggctgtaccagagtgagagtacgggggaggggttgaacaagagttcagggaggagagaattcccagcgctgagccagagactcctttacagaggcccaaggaggcgtggagggagggggaaggctgccaaggctctttctgtctccatgagtgtgtcaagaatgcaaagcactaatgctcttcacttggtccatcttgcagggttgagtttgcagtgagcaaccttgaaggatgagctgacatctcgctcagggccaaataaccgacttgcttactgcttgctataaaatggcacgttacccaaggtcagagttcccttcctataacctccccatccctcacacattcacaggtatctatccaagccatggcatcactctgtggggcttgggggcaaggcaactgacactgcacgctggttctcatgcttgccaagcatgaagccctgtgctgctagcagctgtggaacatagccgttagctttaaaagagggtaaaatcacgtcctggacaggacagccaggtgagttgggaagggaagagagcctgccacgggcacaggcatgttgggggaagtggaagtggtgagagcacagtaggaagtgagaaggggcgggccgtgcttaccaggccgtggacttaaaccagg

SEQ ID NO: 12 - DNA, 635 bp, Artificial Sequence
  Description of Artificial Sequence Synthetic polynucleotide
tgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccg

SEQ ID NO: 13 - DNA, 1205 bp, Artificial Sequence
  Description of Artificial Sequence Synthetic polynucleotide
ctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 14 - DNA, 1843 bp, Artificial Sequence
  Description of Artificial Sequence Synthetic polynucleotide
gagtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 15 - DNA, 1846 bp, Artificial Sequence
  Description of Artificial Sequence Synthetic polynucleotide
ctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccgtgcatcaccgcgttgcggcctcatcagtcccacgactttgtgcccattttactcatgaggagatggaggcccagagagccagtcagaaagtggctgggccaggactaagagtgcagcgcgctgcctccgtgccctgcgtcaacagctcaaggaactggggtgctccggaaatggggccaaggctgctgggcagcaggacgctcagggccttggcctcaggagagcaaattccccactcggagatcggtcttgttgctgcattttattcatgggaaatctgaggctagaagagacgacaaacgacacgccgttggacacacggcaacgttttagatgttgggtctggccgggcggccgtcaccggtcaccatggggaggaggaggagccgagagacttgctcgcggccggggggaggcagaagcgcgtcccgcgggagaggtggctttgaggagtgagctcccggtcccgcggggacgcgagtgggcccagtgcccgggctgccaggcggggcggggcggggccgggcgactgagaggggcggggcctggcggctgggaggggcggggcggatgcggggacagcggcctggctaactcctgcacggcagtgcccttcccggagcgtgccctcgccgggatcc

SEQ ID NO: 16 - DNA, 1591 bp, Artificial Sequence
  Description of Artificial Sequence Synthetic polynucleotide
taggtggcttcacccctctgcctgagcctgagtcctgtccctgccaagactccgcccagccgacgcccaccccagctttccctggactcatccctcagcagatatctggatcctgcctagcctggctcagcatgactcatcatgcagggtaccgcccctgcccacctgttccccaataccgcaattcaggagctgggcagttccccagaggccctaggaaactccccgcccccgaccaggctttctccactcctcccatctgaccgcctgttttctacgcctcacgaccctctgagccccttggcgcactccgacataaccacagccaggcctgagaagccgccagcctccgcagcgagtgtgagcacgggactcagaactggcttctgcacagtgaggacgggacgcggagggggcagcgggaacacgccgcccgcatggctgcgacagttggcagcgccgcgggacagagggaaactgaggccggagccgcagactggacacccgagggggcgacccggggcagcacttggggctcggctacgcgcacagggggcggcgggcagcagagtctgggcctccgcggccggggttccaccgccggccgcctccggctcgcgcaacgggagggaaaacttggacaaccctgccacgcccagcccttggccgcgtggcttctcctgctcgaagcgcggtcccaggagtggccgacgctccctctcctgcccattccgcggatgggcaatcccaggcggaactcccttgagggtctcagaatatctgggagacctcgggctcttgatctccgagacaccccgtttcgtagtggagaacagtccagatcggggaagtttattttgcccaaagccgcatagaggccccctggccctcgattccctctgcggggctcagcagcgttgcagcctagacgggtcttactgtgagccgagcagcctctgggaccacagaccttcccctaccccaacgttagaagccggagcccagcaaggagaagcgcgcacctcctgctgtgaacgcgcacgacgccagggcagctgccagaggccatggcctggcgtgggcctggagcccctctggccagcctgcacggggccagggctacgggataccagcagcgtgccctgggctggatggcaggagagacaggacttgaggctgtcccagaatgggctcaggcagggcgaggatatcaggggaggtggtgtacaggaagcagccgcccagcttgcctggcacacagcaagccctgcccatgaaggcctactgccagaacagtgggcgaggcccggcgtctctgtggagtcggtggggcccgggacagggcagcctgaggcaggtttccactggcggtgaaaggggccgtgtggcaaggacaggagagccagcctcagcccagcaggggaaggcggcccctgagtctccacctggctgctggcagccccactcggagcatcggcgaaactgaggcttgccaaagaagcctttgtccagagtcacgcagctggcgcggtggagccagggccagaacccgtgcaggctgatcccagcctgccttctccactgtgccccg

SEQ ID NO: 17 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 18 - DNA, 770 bp, Homo sapiens
tcttttctgcagttcaagggaaagacgagatcttgcacaaggcactctgcttctgcccttggctggggaagggtggcatggagcctctccggctgctcatcttactctttgtcacagagctgtccggagcccacaacaccacagtgttccagggcgtggcgggccagtccctgcaggtgtcttgcccctatgactccatgaagcactgggggaggcgcaaggcctggtgccgccagctgggagagaagggcccatgccagcgtgtggtcagcacgcacaacttgtggctgctgtccttcctgaggaggtggaatgggagcacagccatcacagacgataccctgggtggcactctcaccattacgctgcggaatctacaaccccatgatgcgggtctctaccagtgccagagcctccatggcagtgaggctgacaccctcaggaaggtcctggtggaggtgctggcagaccccctggatcaccgggatgctggagatctctggttccccggggagtctgagagcttcgaggatgcccatgtggagcacagcatctccaggagcctcttggaaggagaaatccccttcccacccacttccatccttctcctcctggcctgcatctttctcatcaagattctagcagccagcgccctctgggctgcagcctggcatggacagaagccagggacacatccacccagtgaactggactgtggccatgacccagggtatcagctccaaactctgccagggctgagagacacgtga

SEQ ID NO: 19 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAIKDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

(19) United States
(12) Patent Application Publication
(10) Pub. No.: US 2022/0378942 A1
(43) Pub. Date: Dec. 1, 2022

(54) COMPOSITIONS AND METHODS FOR TREATING ALZHEIMER'S DISEASE

(71) Applicants: Children's Medical Center Corporation, Boston, MA (US); Dana-Farber Cancer Institute, Inc., Boston, MA (US)

(72) Inventors: Alessandra BIFFI, Boston, MA (US); Marco PEVIANI, Boston, MA (US)

(73) Assignees: Children's Medical Center Corporation, Boston, MA (US); Dana-Farber Cancer Institute, Inc., Boston, MA (US)

(21) Appl. No.: 17/764,917

(22) PCT Filed: Oct. 1, 2020
(86) PCT No.: PCT/US2020/053824

§ 371 Date: Mar. 29, 2022

Related U.S. Application Data
(60) Provisional application No. 62/908,913, filed on Oct. 1, 2019.

(51) Int. Cl. A61K 48/00; A61K 35/28; C12N 15/10

(52) U.S. Cl. CPC A61K 48/0058; A61K 35/28; C12N 15/10

(57) ABSTRACT

The present disclosure features methods and compositions for treating Alzheimer's disease. The disclosed methods comprise administering to a subject having or suspected of having Alzheimer's a hematopoietic stem progenitor cell expressing at least one neuroprotective agent, such as ApoE2, Trem2, and/or a metallothionein.

### CROSS-REFERENCE TO RELATED APPLICATIONS

This application claims the benefit of the following U.S. Provisional Application No. 62/908,913, filed on Oct. 1, 2019, the entire contents of which are incorporated herein by reference.

### BACKGROUND OF THE INVENTION

Alzheimer's disease is a neurodegenerative disorder characterized by progressive diminished cognitive ability. Recognized as one of the leading causes of death and the leading cause of dementia in the United States, Alzheimer's is a progressive disease with no known cure or effective treatment. Over five million Americans are estimated to have the disease, but it is projected to afflict over fourteen million people by 2060. Neuron loss and extracellular deposition of fibrillar beta-amyloid (Aβ) protein plaques in the brain are prominent pathological features of the disease. AP plaques are also prevalent within and along small and medium-sized arteries and arterioles of the cerebral cortex, meninges, and in the cerebral micro-vasculature, a condition known as cerebral amyloid angiopathy, which is observed in 85% of cases in Alzheimer's disease. As there is no known cure for Alzheimer's diseases, therapeutic methods for treating Alzheimer's disease are urgently required.

### SUMMARY OF THE INVENTION

As described below, the present disclosure features compositions and methods for the treatment of Alzheimer's disease. More specifically, in some embodiments, therapeutic approaches are disclosed that involve administering to a subject in need compositions comprising cells expressing at least one therapeutic transgene.

In one aspect, the invention features an expression vector or expression cassette containing a polynucleotide encoding two or more of an apolipoprotein E isoform 2 (ApoE2) polypeptide, a triggering receptor expressed on myeloid cells 2 (Trem2) polypeptide, and a metallothionein polypeptide, or fragments thereof.

In some embodiments of the above aspect, the polynucleotide encodes ApoE2 and metallothionein 1G, TREM2 and metallothionein 1G, ApoE2 and TREM2, or ApoE2, TREM2 and metallothionein 1G (MT1G).

In another aspect, the invention features an expression vector or expression cassette containing a polynucleotide encoding a TREM2 polypeptide and an ApoE2 polypeptide or fragments thereof.

In any of the above aspects, the vector or cassette contains two or more copies of a metallothionein. In any of the above aspects, the vector contains a polynucleotide encoding at least four copies of MT1G.

In another aspect, the invention features an expression vector containing the expression cassette of any one of the above aspects.

In any of the above aspects, the vector is a lentiviral vector.

In any of the above aspects, the vector contains a promoter driving expression of the polynucleotide. In some embodiments the promoter is the human phosphoglycerate kinase promoter. In some embodiments, the promoter is a microglia specific promoter. In some embodiments, the promoter is a TSPO, MHC class II, or CX3CR1 promoter.

In another aspect, the invention features a lentiviral vector containing a phosphoglycerate kinase (PGK) promoter driving the expression of a polynucleotide encoding a TREM2 polypeptide and an ApoE2 polypeptide, or fragments thereof.

In another aspect, the invention features a lentiviral vector containing a microglia specific promoter driving the expression of a polynucleotide encoding a TREM2 polypeptide and an ApoE2 polypeptide, or fragments thereof.

In any of the above aspects, the vector contains one or more copies of a polynucleotide encoding metallothionein.

In another aspect, the invention provides a cell containing the vector of any of the above aspects or the cassette of any of the above aspects. In various embodiments, the cassette is inserted at a CX3CR1 or a TSPO gene locus. In some embodiments, the cell is a microglial cell or a precursor thereof, a hematopoietic stem cell, a hematopoietic stem progenitor cell (HSPC) or a cell descended from the hematopoietic stem cell or a hematopoietic stem progenitor cell. In some embodiments, the HSPC is CD34⁺ and/or CD38⁻ and/or CD90⁺. In some embodiments, the cell is hemizygous for the CX3CR1 gene.

In another aspect, the invention provides a method of reducing amyloid beta levels in a cell or tissue, the method involving contacting the cell with a polynucleotide encoding two or more of an ApoE2 polypeptide, a Trem2 polypeptide, and a metallothionein polypeptide or fragments thereof.

In another aspect, the invention provides a method of increasing engulfment of beta amyloid by a cell, the method involving contacting the cell with a polynucleotide encoding two or more of an ApoE2 polypeptide, a Trem2 polypeptide, and a metallothionein polypeptide, or fragments thereof.

In another aspect, the invention provides a method of treating a subject having or having a propensity to develop Alzheimer's disease, the method involving administering to the subject an effective amount of a cell containing a polynucleotide encoding two or more of an ApoE2 polypeptide, a Trem2 polypeptide, and a metallothionein polypeptide, or fragments thereof.

In some embodiments, the Alzheimer's disease is familial or early onset Alzheimer's disease.

In another aspect, the invention provides a method of treating a subject having or having a propensity to develop neuroinflammation, the method including administering to the subject an effective amount of a cell containing a polynucleotide encoding two or more of an ApoE2 polypeptide, a Trem2 polypeptide, and a metallothionein polypeptide, or fragments thereof. In any of the above aspects, the polynucleotide is comprised by an expression vector. In some embodiments, the expression vector is a lentiviral vector. In any of the above aspects, the polynucleotide contains an expression cassette. In any of the above aspects, the polynucleotide encodes ApoE2, and TREM2, ApoE2 and metallothionein 1G, TREM2 and metallothionein 1G, or ApoE2, TREM2 and metallothionein 1G (MT1G). In any of the above aspects, the polynucleotide encodes one or more copies of a metallothionein. In any of the above aspects, the polynucleotide encodes at least four copies of MT1G. In any of the above aspects, the polynucleotide encodes a TREM2 polypeptide and an ApoE2 polypeptide or fragments thereof. In any of the above aspects, the polynucleotide further encodes one or more copies of MT1G.

In any of the above aspects the polynucleotide contains a promoter. In any of the above aspects, the polynucleotide contains a constitutive promoter. In some embodiments, the promoter is a phosphoglycerate kinase promoter. In some embodiments, the promoter is a microglial specific promoter. In some embodiments, the promoter is a TSPO, MHC class II, or CX3CR1 promoter.

In any of the above aspects, the cell is a microglial cell or a progenitor thereof, a hematopoietic stem cell, a hematopoietic stem progenitor cell (HSPC), or a cell descended therefrom. In some embodiments the method is carried out in vitro or in vivo. In any of the above aspects, the cell is administered intracerebroventricularly, intravenously, or intrathecally.

In any of the above aspects, the cell is a hematopoietic stem progenitor cell (HSPC). In some embodiments, the HSPC is Lin⁻, CD34⁺, CD38⁻, and/or CD90⁺. In some embodiments, the HSPC is functionally equivalent to a microglial progenitor cell upon transplantation. In some embodiments, the HSPC engrafts in the brain. In some embodiments, the engrafted HSPC is functionally equivalent to or expresses markers characteristic of a microglial progenitor cell.

In any of the above aspects, the subject undergoes ablative conditioning prior to the method. In some embodiments, the ablative conditioning involves administering to the subject an alkylating agent. In some embodiments, the alkylating agent is busulfan. In some embodiments, the conditioning involves administering a CSF-1R inhibitor. In some embodiments, the inhibitor is PLX3397, PLX5622, or liposomal clodronate.

In any of the above aspects, the HSPC is an allogeneic or autologous cell. In any of the above aspects, the cell is hemizygous for the CX3CR1 gene.

In any of the above aspects, the method reduces anxiety, increases cognitive function, or increases short term working memory. In any of the above aspects, the method reduces microglial activation and/or astrocytic response. In any of the above aspects, the method reduces levels of Iba1 and or GFAP.

In another aspect, the invention provides a pharmaceutical composition containing the HSPC of claim 15.

In another aspect, the invention provides a kit containing the HSPC of claim 15 and directions for its delivery to a subject.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “ablative conditioning” refers to administering to a subject a composition that destroys endogenous microglia.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, and a 50% or greater change in expression levels.

By “ameliorate” is meant decrease, suppress, attenuate, diminish, arrest, or stabilize the development or progression of a disease.

By “apolipoprotein E isoform 2 (ApoE2) polypeptide” is meant a protein having at least about 85% amino acid sequence identity to GenBank Accession No. ALQ33369.1 or a fragment thereof and having immunomodulatory activity. An exemplary ApoE2 polypeptide sequence is provided below.

>ALQ33369.1 apolipoprotein E isoform 2, partial
[Homo sapiens]
MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVRAKLEEQAQQIRLQAEAFQAR
LKSWFEPLVEDMQRQWAGLVEKVQAAVGTSAAPVPSDNH

By “apolipoprotein E isoform 2 (ApoE2) polynucleotide” is meant a nucleic acid molecule encoding an ApoE2 polypeptide. The ApoE2 gene encodes a membrane protein that mediates binding, internalization, and catabolism of lipoprotein particles. An exemplary ApoE2 polynucleotide sequence is provided below.

>KU177911.1 Homo sapiens apolipoprotein E isoform
2 (APOE) mRNA, partial cds, alternatively spliced
ATGAAGGTTCTGTGGGCTGCGTTGCTGGTCACATTCCTGGCAGGATGCCA
GGCCAAGGTGGAGCAAGCGGTGGAGACAGAGCCGGAGCCCGAGCTGCGCC
AGCAGACCGAGTGGCAGAGCGGCCAGCGCTGGGAACTGGCACTGGGTCGC
TTTTGGGATTACCTGCGCTGGGTGCAGACACTGTCTGAGCAGGTGCAGGA
GGAGCTGCTCAGCTCCCAGGTCACCCAGGAACTGAGGGCGCTGATGGACG
AGACCATGAAGGAGTTGAAGGCCTACAAATCGGAACTGGAGGAACAACTG
ACCCCGGTGGCGGAGGAGACGCGGGCACGGCTGTCCAAGGAGCTGCAGGC
GGCGCAGGCCCGGCTGGGCGCGGACATGGAGGACGTGCGCGCCAAGCTGG
AGGAGCAGGCCCAGCAGATACGCCTGCAGGCCGAGGCCTTCCAGGCCCGC
CTCAAGAGCTGGTTCGAGCCCCTGGTGGAAGACATGCAGCGCCAGTGGGC
CGGGCTGGTGGAGAAGGTGCAGGCTGCCGTGGGCACCAGCGCCGCCCCTG
TGCCCAGCGACAATCAC

By “biologic sample” is meant any tissue, cell, fluid, or other material derived from an organism.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include neurodegenerative diseases such as Alzheimer's Disease.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In particular embodiments, an effective amount is an amount that reduces at least one symptom of Alzheimer's disease, increases cognitive function, or increases survival.

By “enhancer” is meant a polynucleotide that increases transcription of a gene of interest. In one embodiment, the enhancer comprises 50-1,500 nucleotides. Exemplary enhancers useful in the methods of the invention include, but are not limited to the following:

>MPP05A(hTSPO_upstream_enhancer) (“E1.1”)
agactctgtctcaaaaaaaaaaaaaaaaaaaaaaaaagagagagagaaaa
aggaagttagaaaaacagccctagaggccctacattctgagtaataggag
ttccagaaaggaagtgattgctgcacaacataaatttgaaaagaaagaga
agtgagaaaatagagggaaggaaatcaaagaaataatccaacttctgaaa
agtaaagaatgagcttccagcgggaaagtgcctgttgagtgcaggcacag
tggaggaaatgaagctgggtgtgttgccaggagttggaaacttgtctgag
caacatagcgcgaccctgtgtctacaaaaaaataaaaacaaaacaaaaaa
caaccaaagacttccgaaacagaatggctttagcctgctcaaccgcacac
tggcacctggccaacagcatctcttcatgattctgaaggacaacgatctg
cagctcagccaagcatcagccatctatggcctaggatgcaagaattcagc
aatgttaccttc
>MPP05B(hTSPO_upstream_enhancer) (“E1.2”)
agactctgtctcaaaaaaaaaaaaaaaaaaaaaaaaagagagagagaaaa
aggaagttagaaaaacagccctagaggccctacattctgagtaataggag
ttccagaaaggaagtgattgctgcacaacataaatttgaaaagaaagaga
agtgagaaaatagagggaaggaaatcaaagaaataatccaacttctgaaa
agtaaagaatgagcttccagcgggaaagtgcctgttgagtgcaggcacag
tggaggaaatgaagctgggtgtgttgccaggagttggaaacttgtctgag
caacatagcgcgaccctgtgtctacaaaaaaataaaaacaaaacaaaaaa
caaccaaagacttccgaaacagaatggctttagcctgctcaaccgcacac
tggcacctggccaacagcatctcttcatgattctgaaggacaacgatctg
cagctcagccaagcatcagccatctatggcctaggatgcaagaattcagc
aatgttaccttctgcatcaccgcgttgcggcctcatcagtcccacgactt
tgtgcccattttactcatgaggagatggaggcccagagagccagtcagaa
agtggctgggccaggactaagagtgcagcgcgctgcctccgtgccctgcg
tcaacagctcaaggaactggggtgctccggaaatggggccaaggctgctg
ggcagcaggacgctcagggccttggcctcaggagagcaaattccccactc
ggagatcggtcttgttgctgcattttattcatgggaaatctgaggctaga
agagacgacaaacgacacgccgttggacacacggcaacgttttagatgtt
gggtctggccgggcggccgtcaccggtcaccatggggaggaggaggagcc
gagagacttgctcgcggccggggggaggcagaagcgcgtcccgcgggaga
ggtggctttgaggagtgagctcccggtcccgcggggacgcgagtgggccc
agtgcccgggctgccaggcggggcggggcggggccgggcgactgagaggg
gcggggcctggcggctgggaggggCGGGGCGGATGCGGGGACAGCGGCCT
GGCTAACTCCTGCACGGCAGTGCCCTTCCCGGAGCGTGCCCTCGCCG
>MPP06(hTSPO_intronic_enhancer) (“E2”)
Taggtggcttcacccctctgcctgagcctgagtcctgtccctgccaagac
tccgcccagccgacgcccaccccagctttccctggactcatccctcagca
gatatctggatcctgcctagcctggctcagcatgactcatcatgcagggt
accgcccctgcccacctgttccccaataccgcaattcaggagctgggcag
ttccccagaggccctaggaaactccccgcccccgaccaggctttctccac
tcctcccatctgaccgcctgttttctacgcctcacgaccctctgagcccc
ttggcgcactccgacataaccacagccaggcctgagaagccgccagcctc
cgcagcgagtgtgagcacgggactcagaactggctt

In various embodiments the enhancer is E1, which contains E1.1 and E1.2. In various embodiments the E1 enhancer comprises the sequences E1.1+E1.2 or E1.2+E1.1 in the indicated order from 5′ to 3′.

Sequences comprising other regulatory elements useful in the methods of the invention follow:

>MPP03(hTSPO_upstream_enhancer_plus_upstream_and_intronic_prom)
agactctgtctcaaaaaaaaaaaaaaaaaaaaaaaaagagagagagaaaaaggaagttagaaaaacagccctagaggccctacattctg
agtaataggagttccagaaaggaagtgattgctgcacaacataaatttgaaaagaaagagaagtgagaaaatagagggaaggaaatcaaa
gaaataatccaacttctgaaaagtaaagaatgagcttccagcgggaaagtgcctgttgagtgcaggcacagtggaggaaatgaagctgggt
gtgttgccaggagttggaaacttgtctgagcaacatagcgcgaccctgtgtctacaaaaaaataaaaacaaaacaaaaaacaaccaaagac
ttccgaaacagaatggctttagcctgctcaaccgcacactggcacctggccaacagcatctcttcatgattctgaaggacaacgatctgcag
ctcagccaagcatcagccatctatggcctaggatgcaagaattcagcaatgttaccttcGAGtgcatcaccgcgttgcggcctcatcagtc
ccacgactttgtgcccattttactcatgaggagatggaggcccagagagccagtcagaaagtggctgggccaggactaagagtgcagcgc
gctgcctccgtgccctgcgtcaacagctcaaggaactggggtgctccggaaatggggccaaggctgctgggcagcaggacgctcaggg
ccttggcctcaggagagcaaattccccactcggagatcggtcttgttgctgcattttattcatgggaaatctgaggctagaagagacgacaa
acgacacgccgttggacacacggcaacgttttagatgttgggtctggccgggcggccgtcaccggtcaccatggggaggaggaggagcc
gagagacttgctcgcggccggggggaggcagaagcgcgtcccgcgggagaggtggctttgaggagtgagctcccggtcccgcgggg
acgcgagtgggcccagtgcccgggctgccaggcggggcggggcggggccgggcgactgagaggggcggggcctggcggctggga
ggggCGGGGCGGATGCGGGGACAGCGGCCTGGCTAACTCCTGCACGGCAGTGCCCTT
CCCGGAGCGTGCCCTCGCCGCTGcacagtgaggacgggacgcggagggggcagcgggaacacgccgcccgcat
ggctgcgacagttggcagcgccgcgggacagagggaaactgaggccggagccgcagactggacacccgagggggcgacccgggg
cagcacttggggctcggctacgcgcacagggggcggcgggcagcagagtctgggcctccgcggccggggttccaccgccggccgcc
tccggctcgcgcaacgggagggaaaacttggacaaccctgccacgcccagcccttggccgcgtggcttctcctgctcgaagcgcggtcc
caggagtggccgacgctccctctcctgcccattccgcggatgggcaatcccaggcggaactcccttgagggtctcagaatatctgggaga
cctcgggctcttgatctccgagacaccccgtttcgtagtggagaacagtccagatcggggaagtttattttgcccaaagccgcatagaggcc
ccctggccctcgattccctctgcggggctcagcagcgttgcagcctagacgggtcttactgtgagccgagcagcctctgggaccacagac
cttcccctaccccaacgttagaagccggagcccagcaaggagaagcgcgcacctcctgctgtgaacgcgcacgacgccagggcagctg
ccagaggccatggcctggcgtgggcctggagcccctctggccagcctgcacggggccagggctacgggataccagcagcgtgccctg
ggctggatggcaggagagacaggacttgaggctgtcccagaatgggctcaggcagggcgaggatatcaggggaggtggtgtacagga
agcagccgcccagcttgcctggcacacagcaagccctgcccatgaaggcctactgccagaacagtgggcgaggcccggcgtctctgtg
gagtcggtggggcccgggacagggcagcctgaggcaggtttccactggcggtgaaaggggccgtgtggcaaggacaggagagccag
cctcagcccagcaggggaaggcggcccctgagtctccacctggctgctggcagccccactcggagcatcggcgaaactgaggcttgcc
aaagaagcctttgtccagagtcacgcagctggcgcggtggagccagggccagaacccgtgcaggctgatcccagcctgccttctccactg
tgccccg
>MPP06-01 (hTSPO_prximal5′prom_plus_proximal_upstream_promoter)
Taggtggcttcacccctctgcctgagcctgagtcctgtccctgccaagactccgcccagccgacgcccaccccagctttccctggactcat
ccctcagcagatatctggatcctgcctagcctggctcagcatgactcatcatgcagggtaccgcccctgcccacctgttccccaataccgca
attcaggagctgggcagttccccagaggccctaggaaactccccgcccccgaccaggctttctccactcctcccatctgaccgcctgttttc
tacgcctcacgaccctctgagccccttggcgcactccgacataaccacagccaggcctgagaagccgccagcctccgcagcgagtgtgag
cacgggactcagaactggctttgcatcaccgcgttgcggcctcatcagtcccacgactttgtgcccattttactcatgaggagatggaggcc
cagagagccagtcagaaagtggctgggccaggactaagagtgcagcgcgctgcctccgtgccctgcgtcaacagctcaaggaactgggg
tgctccggaaatggggccaaggctgctgggcagcaggacgctcagggccttggcctcaggagagcaaattccccactcggagatcggtc
ttgttgctgcattttattcatgggaaatctgaggctagaagagacgacaaacgacacgccgttggacacacggcaacgttttagatgttggg
tctggccgggcggccgtcaccggtcaccatggggaggaggaggagccgagagacttgctcgcggccggggggaggcagaagcgcgt
cccgcgggagaggtggctttgaggagtgagctcccggtcccgcggggacgcgagtgggcccagtgcccgggctgccaggcggggcg
gggcggggccgggcgactgagaggggcggggcctggcggctgggaggggCGGGGCGGATGCGGGGACAGCG
GCCTGGCTAACTCCTGCACGGCAGTGCCCTTCCCGGAGCGTGCCCTCGCCG
>MPP03(hTSPO_upstream_plus_intronic_prom)
taggtggcttcacccctctgcctgagcctgagtcctgtccctgccaagactccgcccagccgacgcccaccccagctttccctggactcatc
cctcagcagatatctggatcctgcctagcctggctcagcatgactcatcatgcagggtaccgcccctgcccacctgttccccaataccgcaat
tcaggagctgggcagttccccagaggccctaggaaactccccgcccccgaccaggctttctccactcctcccatctgaccgcctgttttcta
cgcctcacgaccctctgagccccttggcgcactccgacataaccacagccaggcctgagaagccgccagcctccgcagcgagtgtgagc
acgggactcagaactggcttGAGtgcatcaccgcgttgcggcctcatcagtcccacgactttgtgcccattttactcatgaggagatggag
gcccagagagccagtcagaaagtggctgggccaggactaagagtgcagcgcgctgcctccgtgccctgcgtcaacagctcaaggaact
ggggtgctccggaaatggggccaaggctgctgggcagcaggacgctcagggccttggcctcaggagagcaaattccccactcggagat
cggtcttgttgctgcattttattcatgggaaatctgaggctagaagagacgacaaacgacacgccgttggacacacggcaacgttttagatgt
tgggtctggccgggcggccgtcaccggtcaccatggggaggaggaggagccgagagacttgctcgcggccggggggaggcagaag
cgcgtcccgcgggagaggtggctttgaggagtgagctcccggtcccgcggggacgcgagtgggcccagtgcccgggctgccaggcg
gggcggggcggggccgggcgactgagaggggcggggcctggcggctgggaggggCGGGGCGGATGCGGGGACA
GCGGCCTGGCTAACTCCTGCACGGCAGTGCCCTTCCCGGAGCGTGCCCTCGCCGCTG
cacagtgaggacgggacgcggagggggcagcgggaacacgccgcccgcatggctgcgacagttggcagcgccgcgggacagagg
gaaactgaggccggagccgcagactggacacccgagggggcgacccggggcagcacttggggctcggctacgcgcacagggggcg
gcgggcagcagagtctgggcctccgcggccggggttccaccgccggccgcctccggctcgcgcaacgggagggaaaacttggacaac
cctgccacgcccagcccttggccgcgtggcttctcctgctcgaagcgcggtcccaggagtggccgacgctccctctcctgcccattccgcg
gatgggcaatcccaggcggaactcccttgagggtctcagaatatctgggagacctcgggctcttgatctccgagacaccccgtttcgtagtg
gagaacagtccagatcggggaagtttattttgcccaaagccgcatagaggccccctggccctcgattccctctgcggggctcagcagcgtt
gcagcctagacgggtcttactgtgagccgagcagcctctgggaccacagaccttcccctaccccaacgttagaagccggagcccagcaa
ggagaagcgcgcacctcctgctgtgaacgcgcacgacgccagggcagctgccagaggccatggcctggcgtgggcctggagcccctc
tggccagcctgcacggggccagggctacgggataccagcagcgtgccctgggctggatggcaggagagacaggacttgaggctgtcc
cagaatgggctcaggcagggcgaggatatcaggggaggtggtgtacaggaagcagccgcccagcttgcctggcacacagcaagccct
gcccatgaaggcctactgccagaacagtgggcgaggcccggcgtctctgtggagtcggtggggcccgggacagggcagcctgaggca
ggtttccactggcggtgaaaggggccgtgtggcaaggacaggagagccagcctcagcccagcaggggaaggcggcccctgagtctcc
acctggctgctggcagccccactcggagcatcggcgaaactgaggcttgccaaagaagcctttgtccagagtcacgcagctggcgcggt
ggagccagggccagaacccgtgcaggctgatcccagcctgccttctccactgtgccccg

“Exogenous nucleic acid molecule,” as used herein, refers to a nucleic acid molecule that is not an endogenous nucleic acid molecule, i.e., it is a nucleic acid molecule that does not naturally occur in a cell.

By “expression cassette” is meant those vector elements needed for expression of a gene. In one embodiment, an expression cassette comprises a promoter, a polynucleotide encoding a polypeptide of interest, and a terminator.

By “fragment” is meant a portion of a protein or nucleic acid that is substantially identical to a reference protein or nucleic acid. In some embodiments the portion retains at least 50%, 75%, or 80%, 85%, 90%, 95%, or even 99% of the biological activity of the reference protein or nucleic acid described herein.

By “gene locus” is meant a position within a genome where a particular gene sequence is disposed.

By “hematopoietic stem cell (HSC)” is meant a stem cell that gives rise to a variety of blood cells.

By “hematopoietic stem progenitor cell (HSPC)” is meant a cell that gives rise to a hematopoietic stem cell.

Cells (e.g., HSCs, HSPCs) that may be used in conjunction with the compositions and methods described herein include CD34+ cells, CD34+/CD90+ cells, CD34+CD38− cells and CD34+/CD164+ cells. These cells may contain a higher percentage of HSCs or HSPCs. These cells are described in WO2015/059674, WO2017/218948, Radtke et al. Sci. Transl. Med. 9: 1-10, 2017, Radtke et al. Mol Ther Methods Clin Dev. 18:679-691, 2020, and Pellin et al. Nat. Comm. 10: 2395, 2019, the disclosures of each of which are hereby incorporated by reference in their entirety.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Markers of Alzheimer's disease include, but are not limited to, tau protein and beta-amyloid peptide

By “metallothionein 1G (MT1G) polypeptide” is meant a protein having at least about 85% amino acid sequence identity to UniProt Accession No. P13640-1 or a fragment thereof and having heavy metal binding activity. An exemplary MT1G polypeptide sequence is provided below.

>sp|P13640|MT1G_HUMAN Metallothionein-1G OS =
Homo sapiens OX = 9606 GN = MT1G PE = 1 SV = 2
MDPNCSCAAAGVSCTCASSCKCKECKCTSCKKSCCSCCPVGCAKCAQGCI
CKGASEKCSCCA

By “metallothionein 1G (MT1G) polynucleotide” is meant a nucleic acid molecule encoding an MT1G polypeptide. The MT1G gene encodes a protein that binds heavy metals. An exemplary MT1G polynucleotide sequence is provided below.

>NM_005950.2 Homo sapiens metallothionein 1G
(MT1G), transcript variant 1, mRNA
ACTCCGCCTTCCACGTGCACCCACTGCCTCTTCCCTTCTCGCTTGGGAAC
TCTAGTCTCGCCTCGGGTTGCAATGGACCCCAACTGCTCCTGTGCCGCTG
GTGTCTCCTGCACCTGCGCCAGCTCCTGCAAGTGCAAAGAGTGCAAATGC
ACCTCCTGCAAGAAGAGCTGCTGCTCCTGCTGCCCTGTGGGCTGTGCCAA
GTGTGCCCAGGGCTGCATCTGCAAAGGGGCATCGGAGAAGTGCAGCTGCT
GCGCCTGATGTCGGGACAGCCCTGCTCCCAAGTACAAATAGAGTGACCCG
TAAAATCCAGGATTTTTTGTTTTTTGCTACAATCTTGACCCCTTTGCTAC
ATTCCTTTTTTTCTGTGAAATATGTGAATAATAATTAAACACTTAGACTT
GAAAAAAAAAAAAAAAAAAA

By “microglia” is meant an immune cell of the central nervous system.

By “nanoparticle” is meant a composite structure of nanoscale dimensions. In particular, nanoparticles are typically particles of a size in the range of from about 1 to about 1000 nm, and are usually spherical although different morphologies are possible depending on the nanoparticle composition. The portion of the nanoparticle contacting an environment external to the nanoparticle is generally identified as the surface of the nanoparticle. In nanoparticles herein described, the size limitation can be restricted to two dimensions and so that nanoparticles herein described include composite structure having a diameter from about 1 to about 1000 nm, where the specific diameter depends on the nanoparticle composition and on the intended use of the nanoparticle according to the experimental design. For example, nanoparticles to be used in several therapeutic applications typically have a size of about 200 nm or below, and the ones used, in particular, for delivery associated with therapeutic agents typically have a diameter from about 1 to about 100 nm.

As used herein “neurodegenerative disease” refers to any of a group of diseases characterized by the progressive loss of structure and/or function of neurons, including death of neurons. Exemplary neurodegenerative diseases include, without limitation, Alzheimer's disease.

As used herein, “obtaining” as in “obtaining an agent” includes synthesizing, purchasing, or otherwise acquiring the agent.

By “PLX3397” is meant a colony stimulating factor-1 receptor (CSF-1R) inhibitor having the structure

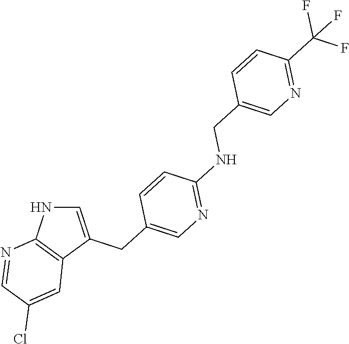 [SMILES: FC(F)(F)c1ccc(CNc2ccc(Cc3c[nH]c4ncc(Cl)cc34)cn2)cn1]

By “PLX5622” is meant a colony stimulating factor-1 receptor (CSF-1R) inhibitor having the structure

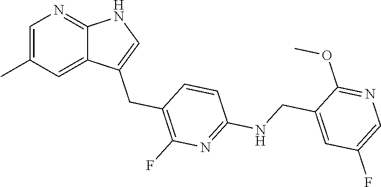 [SMILES: COc1ncc(F)cc1CNc1ccc(Cc2c[nH]c3ncc(C)cc23)c(F)n1]

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “promoter” is meant a polynucleotide sufficient to direct transcription. In some embodiments, the promoter is a translocator protein promoter (TSPO). In some embodiments, the promoter is a CX3CR1 promoter. In an exemplary embodiment, the CX3CR1 promoter comprises a sequence with at least 85% sequence identity to the sequence of GeneBank Accession number GQ258357.1 or a fragment thereof. The sequence of GeneBank Accession number GQ258357.1 is provided below.

GeneBank Accession number GQ258357.1
GCAACCTCCACCTTCCGGTTTCAAGCGATTCTCCTGCCTCAGCCTCCCAAGTAGTTG
GGATTACAGGCACCCGCCACCACGCCTGGCTAATTTTTATATTTTTAGTAAAGACAG
GGTTTCACCATTTTGGCCAGGCTGGTCTTGAACCCCTGATCTCGTGATCCACCCACGT
TGGCCTCCCAAAGTGCTGGAATTACAGGCGTGAGCCACCATGCCTGGCCCACATTAC
ATTCTTACTCACCTCCCCCTACCATGGAATTTTATTCCACAGATATGCTATTGGTTTA
GCTACTATATGTATATCTGTGTTTTATACATAAAGCACAAGAACCTTCCAGAACCAA
TTTTCGCCACCTTGGAAGTAATACCACCTCTACTAAGAATGCACAGCATAGACCATA
AAACCTCAATGCTAAGTTCAAATATTGGCCCTACCACACATGAGCTGTGTGGTCTTG
TACAAGTTACATAACTTCTCCTCCTTGTCTCAAACTCCTCACATATAAGATGAGGAT
AATAATAGTACCTGCGGCCACACACAGTGGCTTAAACGTGTAATCCCAGCACTTTGG
GAGGCTGCGGCAGAAGGATCACTCAAACTCAGGAGTTCAAGAGCAGCCTGGGTACA
TGGCGAAACTCTGTCTCTACAAAAAATACAAAAATTAGCTGGGTGTGGTGATGTGTG
CCTGTAGTTCCAGCTACTTGGGAGGCTGAGGTGAGAGGATCGTTCGAGCCCAGGAG
ATCAAGGATGCAGTGAGCTATGATCATGTGGCTGCACTCCAGCCTGGATAACAGAG
CCAGACCCTGTCTGAAAGAAACAAAAACAAAAACATTAGCACCTGCATCATAGGGT
CACTGGGGGCACTACATGAGTTCATGTACATCGAGGACTTAGGACATTGCCTCAGGC
AGACCTAGTGCTGCACAATTGCTTATGTAATTATTCCCAAATTTCTCCAGGGCCCAC
AGAAGAACATGGAAGTATCTTGGTTTGGCAATTAAGGTGAATCACATTCTCACTCTC
CTTTTCTGCATCTCTACCCCACATTCCCACAAAGCTTTATTCACACCAAGTCTCCAGT
CCTTGCCTGCATTGTGTGATGGGTGCCTGCAGTGATGGGTGGGGACACCCATCACTG
TCCAGGGCGTCCCCACCATCCTCACAGCCTCTCTGTCTGGCCTCCTGCCTTTGAGCCA
GCCCACCACACTCTCATTTCTCTGCCCAGCAGAAACCAAACTGTCCTCTGCATTTACT
GTCTCAACTGGAAGAGAAATGCAGAATGACAAAGAACTTGTGAACAAGGGTCAGCT
CCAACAGAGAGTGAAGCCAAAGGGGCTGGGCAGAAAGAGAGATGAAGACGGGGGT
CTGAGGAATAAGGCTGTACCAGAGTGAGAGTACGGGGGAGGGGTTGAACAAGAGT
TCAGGGAGGAGAGAATTCCCAGCGCTGAGCCAGAGACTCCTTTACAGAGGCCCAAG
GAGGCGTGGAGGGAGGGGGAAGGCTGCCAAGGCTCTTTCTGTCTCCATGAGTGTGT
CAAGAATGCAAAGCACTAATGCTCTTCACTTGGTCCATCTTGCAGGGTTGAGTTTGC
AGTGAGCAACCTTGAAGGATGAGCTGACATCTCGCTCAGGGCCAAATAACCGACTT
GCTTACTGCTTGCTATAAAATGGCACGTTACCCAAGGTCAGAGTTCCCTTCCTATAA
CCTCCCCATCCCTCACACATTCACAGGTATCTATCCAAGCCATGGCATCACTCTGTG
GGGCTTGGGGGCAAGGCAACTGACACTGCACGCTGGTTCTCATGCTTGCCAAGCAT
GAAGCCCTGTGCTGCTAGCAGCTGTGGAACATAGCCGTTAGCTTTAAAAGAGGGTA
AAATCACGTCCTGGACAGGACAGCCAGGTGAGTTGGGAAGGGAAGAGAGCCTGCC
ACGGGCACAGGCATGTTGGGGGAAGTGGAAGTGGTGAGAGCACAGTAGGAAGTGA
GAAGGGGCGGGCCGTGCTTACCAGGCCGTGGACTTAAACCAGG

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be, in some embodiments at least about 16 amino acids, at least about 20 amino acids, at least about 25 amino acids, or about 35 amino acids, about 50 amino acids, or about 100 amino acids, or any integer thereabout or therebetween. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “nucleotide molecule”, “polynucleotide”, or “nucleic acid sequence” are used interchangeably to refer to a molecule comprising RNA or DNA. In various embodiments, the nucleotide molecule or polynucleotide comprises modified nucleotides (e.g., locked nucleic acids (LNA)). In some embodiments, the nucleotide molecule or polynucleotide comprises RNA and DNA. The sugar backbone of the nucleotide molecule is non-limiting and may comprise ribose, deoxyribose, or various other suitable sugars. In some embodiments, the nucleic acid molecule comprises at least two nucleotides covalently linked together. In some embodiments, the nucleic acid molecule of the present invention is single-stranded. In some embodiments, the nucleic acid molecule is double stranded. In some embodiments, the nucleic acid molecule is triple-stranded. In some embodiments, the nucleic acid molecule comprises phosphodiester bonds. In some embodiments, the nucleic acid molecule comprises a single-stranded or double-stranded deoxyribonucleic acid (DNA) or a single-stranded or double-stranded ribonucleic acid (RNA). In some embodiments, the nucleic acid molecule comprises a nucleic acid analog. In some embodiments, the nucleic acid analog has a backbone, comprising a bond other than and/or in addition to a phosphodiester bond, such as, by non-limiting example, phosphoramide, phosphorothioate, phosphorodithioate or O-methylphophoroamidite linkage. In some embodiments, the nucleic acid analog is selected from a nucleic acid analog with a backbone selected from a positive backbone; a non-ionic backbone and a non-ribose backbone. In some embodiments, the nucleic acid molecule contains one or more carbocyclic sugars. In some embodiments, the nucleic acid molecule comprises modifications of its ribose-phosphate backbone. In some embodiments, these modifications are performed to facilitate the addition of additional moieties, such as labels. In some embodiments, these modifications are performed to increase the stability and half-life of such molecules in physiological environments. In some embodiments, the term “polynucleotide” captures sequences that include any of the known base analogues of DNA and RNA such as, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

For example, stringent salt concentration will ordinarily be less than about 750 mM NaCl and 75 mM trisodium citrate, less than about 500 mM NaCl and 50 mM trisodium citrate, or about 250 mM NaCl and 25 mM trisodium citrate. Low stringency hybridization can be obtained in the absence of organic solvent, e.g., formamide, while high stringency hybridization can be obtained in the presence of at least about 35% formamide, and in some embodiments, at least about 50% formamide. Stringent temperature conditions will ordinarily include temperatures of at least about 30° C. at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will comprise less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In some embodiments, wash steps will occur at 25° C. in 30 mM NaCl, 3 mM trisodium citrate, and 0.1% SDS. In other embodiments, wash steps will occur at 42 C in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

By “translocator protein promoter” or “TSPO promoter” is meant a polynucleotide sufficient to direct expression of a transgene in a microglial cell. In one embodiment the TSPO promoter is responsive to inflammation. Exemplary promoters useful in the methods of the invention include, but are not limited to, the following:

>MPP01(hTSPO_prximal5′prom)(“P1”)
Tgcatcaccgcgttgcggcctcatcagtcccacgactttgtgcccatttt
actcatgaggagatggaggcccagagagccagtcagaaagtggctgggcc
aggactaagagtgcagcgcgctgcctccgtgccctgcgtcaacagctcaa
ggaactggggtgctccggaaatggggccaaggctgctgggcagcaggacg
ctcagggccttggcctcaggagagcaaattccccactcggagatcggtct
tgttgctgcattttattcatgggaaatctgaggctagaagagacgacaaa
cgacacgccgttggacacacggcaacgttttagatgttgggtctggccgg
gcggccgtcaccggtcaccatggggaggaggaggagccgagagacttgct
cgcggccggggggaggcagaagcgcgtcccgcgggagaggtggctttgag
gagtgagctcccggtcccgcggggacgcgagtgggcccagtgcccgggct
gccaggcggggcggggcggggccgggcgactgagaggggcggggcctggc
ggctgggaggggCGGGGCGGATGCGGGGACAGCGGCCTGGCTAACTCCTG
CACGGCAGTGCCCTTCCCGGAGCGTGCCCTCGCCG

The P1 promoter comprises 635 bp that correspond to nucleotide residues −562 to +73 (capital letters) of the hTspo immediate 5′ promoter of the hTspo immediate 5′ promoter.

>MPP02(hTSPO_intronic5′prom)(“P2”)
CTGcacagtgaggacgggacgcggagggggcagcgggaacacgccgcccgcatggctgcgacagttggcagcgccgcgggaca
gagggaaactgaggccggagccgcagactggacacccgagggggcgacccggggcagcacttggggctcggctacgcgcacaggg
ggcggcgggcagcagagtctgggcctccgcggccggggttccaccgccggccgcctccggctcgcgcaacgggagggaaaacttgg
acaaccctgccacgcccagcccttggccgcgtggcttctcctgctcgaagcgcggtcccaggagtggccgacgctccctctcctgcccatt
ccgcggatgggcaatcccaggcggaactcccttgagggtctcagaatatctgggagacctcgggctcttgatctccgagacaccccgtttc
gtagtggagaacagtccagatcggggaagtttattttgcccaaagccgcatagaggccccctggccctcgattccctctgcggggctcagc
agcgttgcagcctagacgggtcttactgtgagccgagcagcctctgggaccacagaccttcccctaccccaacgttagaagccggagccc
agcaaggagaagcgcgcacctcctgctgtgaacgcgcacgacgccagggcagctgccagaggccatggcctggcgtgggcctggagc
ccctctggccagcctgcacggggccagggctacgggataccagcagcgtgccctgggctggatggcaggagagacaggacttgaggc
tgtcccagaatgggctcaggcagggcgaggatatcaggggaggtggtgtacaggaagcagccgcccagcttgcctggcacacagcaag
ccctgcccatgaaggcctactgccagaacagtgggcgaggcccggcgtctctgtggagtcggtggggcccgggacagggcagcctga
ggcaggtttccactggcggtgaaaggggccgtgtggcaaggacaggagagccagcctcagcccagcaggggaaggcggcccctgag
tctccacctggctgctggcagccccactcggagcatcggcgaaactgaggcttgccaaagaagcctttgtccagagtcacgcagctggcg
cggtggagccagggccagaacccgtgcaggctgatcccagcctgccttctccactgtgccccg
>MPP03(hTSPO_upstream_plus_intronic_prom)(“P1+P2”)
GAGtgcatcaccgcgttgcggcctcatcagtcccacgactttgtgcccattttactcatgaggagatggaggcccagagagccagtcaga
aagtggctgggccaggactaagagtgcagcgcgctgcctccgtgccctgcgtcaacagctcaaggaactggggtgctccggaaatgggg
ccaaggctgctgggcagcaggacgctcagggccttggcctcaggagagcaaattccccactcggagatcggtcttgttgctgcattttatt
catgggaaatctgaggctagaagagacgacaaacgacacgccgttggacacacggcaacgttttagatgttgggtctggccgggcggcc
gtcaccggtcaccatggggaggaggaggagccgagagacttgctcgcggccggggggaggcagaagcgcgtcccgcgggagaggt
ggctttgaggagtgagctcccggtcccgcggggacgcgagtgggcccagtgcccgggctgccaggcggggcggggcggggccggg
cgactgagaggggcggggcctggcggctgggaggggCGGGGCGGATGCGGGGACAGCGGCCTGGCTA
ACTCCTGCACGGCAGTGCCCTTCCCGGAGCGTGCCCTCGCCGCTGcacagtgaggacgggacg
cggagggggcagcgggaacacgccgcccgcatggctgcgacagttggcagcgccgcgggacagagggaaactgaggccggagcc
gcagactggacacccgagggggcgacccggggcagcacttggggctcggctacgcgcacagggggcggcgggcagcagagtctgg
gcctccgcggccggggttccaccgccggccgcctccggctcgcgcaacgggagggaaaacttggacaaccctgccacgcccagccctt
ggccgcgtggcttctcctgctcgaagcgcggtcccaggagtggccgacgctccctctcctgcccattccgcggatgggcaatcccaggcgg
aactcccttgagggtctcagaatatctgggagacctcgggctcttgatctccgagacaccccgtttcgtagtggagaacagtccagatcgg
ggaagtttattttgcccaaagccgcatagaggccccctggccctcgattccctctgcggggctcagcagcgttgcagcctagacgggtctt
actgtgagccgagcagcctctgggaccacagaccttcccctaccccaacgttagaagccggagcccagcaaggagaagcgcgcacctcct
gctgtgaacgcgcacgacgccagggcagctgccagaggccatggcctggcgtgggcctggagcccctctggccagcctgcacggggc
cagggctacgggataccagcagcgtgccctgggctggatggcaggagagacaggacttgaggctgtcccagaatgggctcaggcagg
gcgaggatatcaggggaggtggtgtacaggaagcagccgcccagcttgcctggcacacagcaagccctgcccatgaaggcctactgcc
agaacagtgggcgaggcccggcgtctctgtggagtcggtggggcccgggacagggcagcctgaggcaggtttccactggcggtgaaa
ggggccgtgtggcaaggacaggagagccagcctcagcccagcaggggaaggcggcccctgagtctccacctggctgctggcagcccc
actcggagcatcggcgaaactgaggcttgccaaagaagcctttgtccagagtcacgcagctggcgcggtggagccagggccagaaccc
gtgcaggctgatcccagcctgccttctccactgtgccccg
>MPP04(hTSPO_intronic_plus_upstream_prom)(“P2+P1”)
CTGcacagtgaggacgggacgcggagggggcagcgggaacacgccgcccgcatggctgcgacagttggcagcgccgcgggaca
gagggaaactgaggccggagccgcagactggacacccgagggggcgacccggggcagcacttggggctcggctacgcgcacaggg
ggcggcgggcagcagagtctgggcctccgcggccggggttccaccgccggccgcctccggctcgcgcaacgggagggaaaacttgg
acaaccctgccacgcccagcccttggccgcgtggcttctcctgctcgaagcgcggtcccaggagtggccgacgctccctctcctgcccatt
ccgcggatgggcaatcccaggcggaactcccttgagggtctcagaatatctgggagacctcgggctcttgatctccgagacaccccgtttc
gtagtggagaacagtccagatcggggaagtttattttgcccaaagccgcatagaggccccctggccctcgattccctctgcggggctcagc
agcgttgcagcctagacgggtcttactgtgagccgagcagcctctgggaccacagaccttcccctaccccaacgttagaagccggagccc
agcaaggagaagcgcgcacctcctgctgtgaacgcgcacgacgccagggcagctgccagaggccatggcctggcgtgggcctggagc
ccctctggccagcctgcacggggccagggctacgggataccagcagcgtgccctgggctggatggcaggagagacaggacttgaggc
tgtcccagaatgggctcaggcagggcgaggatatcaggggaggtggtgtacaggaagcagccgcccagcttgcctggcacacagcaag
ccctgcccatgaaggcctactgccagaacagtgggcgaggcccggcgtctctgtggagtcggtggggcccgggacagggcagcctga
ggcaggtttccactggcggtgaaaggggccgtgtggcaaggacaggagagccagcctcagcccagcaggggaaggcggcccctgag
tctccacctggctgctggcagccccactcggagcatcggcgaaactgaggcttgccaaagaagcctttgtccagagtcacgcagctggcg
cggtggagccagggccagaacccgtgcaggctgatcccagcctgccttctccactgtgccccgtgcatcaccgcgttgcggcctcatcag
tcccacgactttgtgcccattttactcatgaggagatggaggcccagagagccagtcagaaagtggctgggccaggactaagagtgcagc
gcgctgcctccgtgccctgcgtcaacagctcaaggaactggggtgctccggaaatggggccaaggctgctgggcagcaggacgctcag
ggccttggcctcaggagagcaaattccccactcggagatcggtcttgttgctgcattttattcatgggaaatctgaggctagaagagacga
caaacgacacgccgttggacacacggcaacgttttagatgttgggtctggccgggcggccgtcaccggtcaccatggggaggaggaggag
ccgagagacttgctcgcggccggggggaggcagaagcgcgtcccgcgggagaggtggctttgaggagtgagctcccggtcccgcgg
ggacgcgagtgggcccagtgcccgggctgccaggcggggcggggcggggccgggcgactgagaggggcggggcctggcggctg
ggaggggCGGGGCGGATGCGGGGACAGCGGCCTGGCTAACTCCTGCACGGCAGTGCCC
TTCCCGGAGCGTGCCCTCGCCGGGATCC
>MPP02(hTSPO_intronic5′prom)
taggtggcttcacccctctgcctgagcctgagtcctgtccctgccaagactccgcccagccgacgcccaccccagctttccctggactcatc
cctcagcagatatctggatcctgcctagcctggctcagcatgactcatcatgcagggtaccgcccctgcccacctgttccccaataccgcaat
tcaggagctgggcagttccccagaggccctaggaaactccccgcccccgaccaggctttctccactcctcccatctgaccgcctgttttcta
cgcctcacgaccctctgagccccttggcgcactccgacataaccacagccaggcctgagaagccgccagcctccgcagcgagtgtgagc
acgggactcagaactggcttCTGcacagtgaggacgggacgcggagggggcagcgggaacacgccgcccgcatggctgcgacagt
tggcagcgccgcgggacagagggaaactgaggccggagccgcagactggacacccgagggggcgacccggggcagcacttgggg
ctcggctacgcgcacagggggcggcgggcagcagagtctgggcctccgcggccggggttccaccgccggccgcctccggctcgcgc
aacgggagggaaaacttggacaaccctgccacgcccagcccttggccgcgtggcttctcctgctcgaagcgcggtcccaggagtggccg
acgctccctctcctgcccattccgcggatgggcaatcccaggcggaactcccttgagggtctcagaatatctgggagacctcgggctcttga
tctccgagacaccccgtttcgtagtggagaacagtccagatcggggaagtttattttgcccaaagccgcatagaggccccctggccctcgat
tccctctgcggggctcagcagcgttgcagcctagacgggtcttactgtgagccgagcagcctctgggaccacagaccttcccctaccccaa
cgttagaagccggagcccagcaaggagaagcgcgcacctcctgctgtgaacgcgcacgacgccagggcagctgccagaggccatggc
ctggcgtgggcctggagcccctctggccagcctgcacggggccagggctacgggataccagcagcgtgccctgggctggatggcagg
agagacaggacttgaggctgtcccagaatgggctcaggcagggcgaggatatcaggggaggtggtgtacaggaagcagccgcccagc
ttgcctggcacacagcaagccctgcccatgaaggcctactgccagaacagtgggcgaggcccggcgtctctgtggagtcggtggggccc
gggacagggcagcctgaggcaggtttccactggcggtgaaaggggccgtgtggcaaggacaggagagccagcctcagcccagcagg
ggaaggcggcccctgagtctccacctggctgctggcagccccactcggagcatcggcgaaactgaggcttgccaaagaagcctttgtcca
gagtcacgcagctggcgcggtggagccagggccagaacccgtgcaggctgatcccagcctgccttctccactgtgccccg

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “transgene” is meant an exogenous nucleic acid molecule, introduced into a host cell, that encodes a polypeptide or polynucleotide to be expressed in the host cell.

By “triggering receptor expressed on myeloid cells 2 (Trem2) polypeptide” is meant a protein having at least about 85% amino acid sequence identity to UniProt Accession No. Q9NZC2 or a fragment thereof and having immunomodulatory activity. An exemplary Trem2 polypeptide sequence is provided below.

>sp|Q9NZC2|TREM2_HUMAN Triggering receptor
expressed on myeloid cells 2 OS = Homo sapiens
OX = 9606 GN = TREM2 PE = 1 SV= 1
MEPLRLLILLFVTELSGAHNTTVFQGVAGQSLQVSCPYDSMKHWGRRKAW
CRQLGEKGPCQRVVSTHNLWLLSFLRRWNGSTAITDDTLGGTLTITLRNL
QPHDAGLYQCQSLHGSEADTLRKVLVEVLADPLDHRDAGDLWFPGESESF
EDAHVEHSISRSLLEGEIPFPPTSILLLLACIFLIKILAASALWAAAWHG
QKPGTHPPSELDCGHDPGYQLQTLPGLRDT

By “triggering receptor expressed on myeloid cells 2 (TREM2) polynucleotide” is meant a nucleic acid molecule encoding a Trem2 polypeptide. The TREM2 gene encodes a membrane protein that forms a receptor signaling complex with the TYRO protein tyrosine kinase binding protein. An exemplary TREM2 polynucleotide sequence is provided below.

>AK312215.1 Homo sapiens cDNA, FLJ92504, Homo
sapiens triggering receptor expressed on myeloid
cells 2(TREM2), mRNA
TCTTTTCTGCAGTTCAAGGGAAAGACGAGATCTTGCACAAGGCACTCTGC
TTCTGCCCTTGGCTGGGGAAGGGTGGCATGGAGCCTCTCCGGCTGCTCAT
CTTACTCTTTGTCACAGAGCTGTCCGGAGCCCACAACACCACAGTGTTCC
AGGGCGTGGCGGGCCAGTCCCTGCAGGTGTCTTGCCCCTATGACTCCATG
AAGCACTGGGGGAGGCGCAAGGCCTGGTGCCGCCAGCTGGGAGAGAAGGG
CCCATGCCAGCGTGTGGTCAGCACGCACAACTTGTGGCTGCTGTCCTTCC
TGAGGAGGTGGAATGGGAGCACAGCCATCACAGACGATACCCTGGGTGGC
ACTCTCACCATTACGCTGCGGAATCTACAACCCCATGATGCGGGTCTCTA
CCAGTGCCAGAGCCTCCATGGCAGTGAGGCTGACACCCTCAGGAAGGTCC
TGGTGGAGGTGCTGGCAGACCCCCTGGATCACCGGGATGCTGGAGATCTC
TGGTTCCCCGGGGAGTCTGAGAGCTTCGAGGATGCCCATGTGGAGCACAG
CATCTCCAGGAGCCTCTTGGAAGGAGAAATCCCCTTCCCACCCACTTCCA
TCCTTCTCCTCCTGGCCTGCATCTTTCTCATCAAGATTCTAGCAGCCAGC
GCCCTCTGGGCTGCAGCCTGGCATGGACAGAAGCCAGGGACACATCCACC
CAGTGAACTGGACTGTGGCCATGACCCAGGGTATCAGCTCCAAACTCTGC
CAGGGCTGAGAGACACGTGA

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof Δny compositions or methods provided herein can be combined with one or more of any of the other compositions and methods provided herein.

### BRIEF DESCRIPTION OF THE DRAWINGS

FIG. 1 illustrates the expression of transgenes according to the presently disclosed methods. FIG. 1 is an image of a Western blot showing Trem2 and ApoE2 expression in 293T cells and BV-2 microglia cells previously transduced with lentiviral vectors encoding cDNAs of human ApoE2 and Trem2.

FIGS. 2A to 2E are schematics, an annotated sequence, peptide domain maps, and a bar graph relating to lentiviral vector generation and testing. FIG. 2A is a schematic of Trem2 protein structure on the cell membrane. ECS, extracellular space; ICS, intracellular space. FIG. 2B is an annotated amino acid sequence of Trem2. Shaded fragments indicate the functional domains on Trem2 while letters in boldface or that are shaded indicate the Alzheimer's disease-(AD) associated single amino acid mutations on Trem2. FIG. 2C provides peptide domain maps for ApoE variants: ApoE2, ApoE3 and ApoE4. ApoE3 is the dominant “neutral” variant in the majority of people. ApoE4 is associated with the increased occurrence of Alzheimer's disease (AD), while ApoE2 carriers show a certain level of resistance to Alzheimer's disease (AD). FIG. 2D is a schematic of an in vitro model to assess the capability of Aβ engulfment by microglial cell line, BV2 cells, with a fluorescent imaging system. FIG. 2E is a bar graph showing AB oligomer uptake quantification by transduced and control cells. Mean+/−SD. Asterisks indicate significance at Kruskall Wallis test, “*” indicates p<0.05, and “****” indicates p<0.0001.

FIGS. 3A and 3B are a bar graph and Western blot images showing 5×FAD hematopoietic stem cell (HSC) transduction with therapeutic vectors. Lin− hematopoietic stem progenitor cells (HSPCs) were isolated from 5×FAD donors and transduced with the therapeutic ApoE2, Trem2 and MT1G lentiviral vectors (LVs). FIG. 3A is a bar graph presenting transduction efficiency of the 5×FAD HSPCs with the indicated vectors expressed as mean vector copy number (VCN)+/−SD. Average vector copy number was measured in liquid culture progeny of the HSPCs transduced with indicated lentiviral vectors for in vivo transplantation. FIG. 3B provides Western blots demonstrating human ApoE2 and human Trem2 expression in the liquid culture progeny of the transduced HSCs. HSCs were transduced with the indicated lentiviral vectors. Positive control: HepG cells expressing hApoE2.

FIGS. 4A to 4F are bar graphs and plots showing 5×FAD mice phenotypes at behavioral testing. 5×FAD mice (untreated and mock transplanted) and age matched wild type controls (untreated and mock transplanted) underwent monthly behavioral testing from 4 to 12 months of age by novel object recognition test (FIG. 4A), Y maze (FIG. 4B) and elevated plus maze (FIG. 4C); the latter demonstrated a statistically significant behavioral deficit in 5×FAD animals from 6 months of age. At 12 months of age mice underwent Morris Mater Maze test which showed a clear phenotypic different by multiple parameters, including the probe trial (FIG. 4D), the latency to reach the hidden platform (FIG. 4E), and the reverse trial latency (FIG. 4F). Mean+/−SEM (standard error of the mean), asterisks indicate significance at multiple t test for repeated measures.

FIGS. 5A to 5D are bar graphs and plots showing treated 5×FAD mice phenotype at behavioral testing. Treated 5×FAD mice and age matched controls (untreated and mock transplanted) underwent monthly behavioral testing elevated plus maze (FIG. 5A) and Morris water maze-probe trial (FIG. 5B), latency to reach the hidden platform (FIG. 5C), reverse trial latency (FIG. 5D). Mean+/−SEM (standard error of the mean), asterisks indicate significance at Two Ways Anova with Dunnet's post-test versus 5×FAD in FIG. 5A, with Tukey's posttest vs wild type in FIG. 5C and vs 5×FAD in FIG. 5D, and One Way Anova with Tukey's posttest vs WT in FIG. 5B.

FIGS. 6A to 6H are bar graphs showing treated 5×FAD mice phenotype at histological evaluation. Treated 5×FAD mice and age matched controls (untreated and mock transplanted) underwent sacrifice at >12 months of age and their brain processed for staining and signal quantification for Iba1 (FIG. 6A—cortex, FIG. 6B—hippocampus), GFAP (FIG. 6C—cortex, FIG. 6D—hippocampus), Ab (FIG. 6E—cortex, FIG. 6F—hippocampus; FIG. 6G—cortex of mice with vector copy number (VCN)>0.5 in brain, FIG. 6H—hippocampus of mice with vector copy number (VCN)>0.5 in brain). Mean+/−SEM (standard error of the mean), asterisks indicate significance at One Way Anova with Dunnet's post-test versus WT in FIG. 6A and vs 5×FAD in FIG. 611.

### DETAILED DESCRIPTION OF THE INVENTION

The invention features compositions and methods that are useful for reconstituting microglia upon transplantation of HSPCs, as well as for the treatment and prevention of Alzheimer's disease.

As reported in greater detail below, the present invention is based at least in part on the discovery that behavioral symptoms present in a mouse model of Alzheimer's disease were ameliorated following transplantation with HSPCs expressing ApoE2. Accordingly, the invention provides compositions and methods for treating Alzheimer's disease using HSPCs that overexpress ApoE2 and/or other therapeutic agents, such as TREM2 and metallothioneins.

Among other things, the invention provides approaches for effective genetic engineering of central nervous system (CNS) microglia and myeloid cells for delivery of therapeutics to the brain for treatment of neurodegenerative diseases. Microglia replacement after transplantation of genetically modified hematopoietic stem cells (HSCs) in gene therapy clinical trials has demonstrated potential to restrain neural deterioration in monogenic neurodegenerative diseases. Not wishing to be bound by theory, early, during development, a group of myeloid hematopoietic progenitors integrate in the central nervous system (CNS) to provide life-long support. Indeed, recent advances in hematopoietic cell transplantation (HCT) provided evidence that, after myeloablation and infusion of HSCs, new microglia-like cells arise and settle in the central nervous system (CNS). These cells can integrate locally and functionally, and besides providing therapeutic molecules to surrounding cells can potentially positively contribute to the neural environment by defining and reshaping the neuronal network, maintaining neuron homeostasis, pruning synaptic spines, and of course being a functional part of the immune system for both surveillance and phagocytosis. Gene therapy clinical trials have been developed to target monogenic neurodegenerative disorders in which paracrine release of critical lysosomal enzymes demonstrated therapeutic benefit. Thus, engineering microglia via intra-CNS transplantation of genetically modified hematopoietic stem cells (HSCs) could offer a route for medical intervention for currently incurable neurodegenerative disorders, such as amyotrophic lateral sclerosis (ALS) and Alzheimer's disease (AD).

### Therapeutic Agents: ApoE2, TREM2, and Metallothionein

The invention features cells over expressing Apolipoprotein E (ApoE) and Triggering Receptor Expressed on Myeloid Cells 2 (TREM2). ApoE, a major apolipoprotein in the central nervous system (CNS), is generally known to be involved in the lipid metabolism in different organs. However, ApoE is also synthesized and secreted by astrocytes, microglia and to a lesser extent by neurons in the central nervous system. Brain ApoE is involved in injury repair via the redistribution of lipids as well as the modulation of neurite outgrowth and cerebrovascular integrity. ApoE is closely involved in the pathogenesis of late-onset forms of Alzheimer's disease, with the APOE4 allele being associated with a markedly increased risk of developing AD, whereas APOE2 is neuroprotective (reduced AD risk by 50% and delayed disease onset) (Serrano-Pozo et al., Ann Neurol 2015; 10:371). In line with this latter observation, direct CNS-gene delivery of APOE2 in AD mouse models was neuroprotective and decreased Aβ levels and amyloid plaques (Zhao et al., Neurobiol Aging 2016; 44:159).

TREM2 (Triggering receptor expressed on myeloid cells 2) is a microglia cell-surface receptor whose deficiency or haploinsufficiency augments Aβ accumulation due to a dysfunctional response of cells, which become apoptotic (Wang et al., Cell 2015; 160(6):1061) (Guerreiro & Hardy, 2014). Restoring or increasing TREM2 function in microglia is likely to improve the scavenging of neurotoxic Ab in AD. Viral vector mediated up-regulation of TREM2, in the brain of AD mice, if applied at the early phases of disease development, ameliorated AD-related neuropathology including Aβ deposition, neuroinflammation, and neuronal and synaptic loss, which was accompanied by an improvement in spatial cognitive functions (Jiang et al., Neuropsycopharmacology 2014; 39(13):2949).

Interestingly, staining with anti-Aβ antibodies has revealed relatively extensive amyloid deposition in the same areas where elevated TSPO-selective radioligands binding was detected. This is in line with a proposed association between amyloido-pathology and reactive microgliosis. Thus, the invention, among other things, provides a gene therapy strategy based on transplantation of lentivirus vector- (LV) transduced hematopoietic stem progenitor cells (HSPCs), aimed at generating a microglia progeny genetically engineered to express TREM2 and/or APOE2 in a regulated manner, under the control of TSPO promoter. This allows specific release of the therapeutic factors by newly generated microglia recruited in the vicinity of disease sites (amyloid plaques) with potential for more efficient specific scavenging of misfolded proteins at sites of neuronal demise. In various embodiments of the invention, overexpression of TREM2 and/or APOE2 in brain myeloid/microglia progeny derived from transplanted hematopoietic stem progenitor cells (HSPCs) is a valuable alternative to the already tested central nervous system- (CNS) gene delivery approaches, since the invention allows more widespread delivery of the protective factors in the whole brain parenchyma. Metallothioneins (MTs) belong to the group of intracellular cysteine-rich, metal-binding proteins and have been involved in homeostasis of essential metals such as zinc and copper, detoxification of toxic metals such as cadmium, and protection against oxidative stress. Furthermore, the MTs have been implicated in processes of neuroprotection and neuroregeneration in several conditions, including AD (Juarez-Rebollar, Rios, Nava-Ruiz, & Mendez-Armenta, 2017; Ruttkay-Nedecky et al., 2013). In addition, overexpression of metallothioneins (such as MT1G) contributes to neuroprotection in LSD mouse models for infantile neuronal ceroid lipofuscinosis and Krabbe disease. Thus, metallothioneins may be beneficial also for Alzheimer's disease (AD).

### HSC Transplantation in Alzheimer's Disease

Neurodegenerative diseases, such as Alzheimer's disease, are characterized by the progressive loss of functional neurons. In some neurodegenerative diseases, a relationship between microglia residing in the central nervous system and neurodegeneration has been observed. For example, activation of glial cells in the brains of patients plays a role in disease expansion to other regions of the central nervous system, and aberrant activation of microglia cells in Alzheimer's patients can promote a neurotoxic environment, which can contribute to the signature loss of motor neurons.

The present disclosure features compositions comprising hematopoietic stem progenitor cells (HSPCs) over expressing ApoE2 and/or other therapeutic agents, such as TREM2 and metallothioneins, which are useful for transplantation into the brains of subjects suffering from Alzheimer's disease that have undergone ablative conditioning to destroy endogenous microglia. This conditioning improves engraftment of the transplanted HSPCs. In some embodiments, HSPCs are delivered directly to the brain via intracerebroventricular injection (ICV). In other embodiments, HSPCs are provided via intravenous (IV) injection. HSPCs used to reconstitute microglial populations are modified to express ApoE2, TREM2 and/or metallothioneins.

ICV delivery following ablative conditioning is superior to conventional methods for treating neurodegenerative diseases using HSC transplantation, which are generally ineffective because of the slow replacement of resident microglia by the progeny of the transplanted cells. Conventional methods for HSC transplantation include the use of total bone marrow or aphaeretic products or cord blood, or of hematopoietic stem progenitor cells (HSPCs) in the case of autologous gene therapy. In contrast, the present invention provides cell populations enriched for cells having microglia-repopulating activity. Moreover, it has been found that hematopoietic stem progenitor cells (HSPC) or fractions of the HSPC pool delivered directly to the brain using intracerebroventricular injection (ICV) improved the speed and extent of microglia reconstitution by the transplanted donor cells and increased therapeutic protein delivery to the brain as compared to a single intravenous (IV) transplantation. The ICV approach is therapeutically beneficial and enhances microglia replacement by the transplanted cells. Additionally, this disclosure contemplates molecular engineering of microglia for therapeutic gene expression (e.g., expression of ApoE2, TREM2 and/or metallothioneins).

### Regenerating Engineered Microglial Cells in the Central Nervous System

Microglia have a developmental origin distinct from that of bone marrow-derived myelomonocytes (Ginhoux et al. Science 330, 841-845 (2010)) the contents of which are incorporated herein by reference in their entirety). However, cells having a microglia-like phenotype can be derived from transplanted donor HSPCs. HSPCs capable generating microglia-like cells upon transplantation into myeloablated recipients are retained within human and murine long-term hematopoietic stem cells (HSCs), thereby providing a reservoir of pluripotent cells capable of differentiating into therapeutic microglia for the treatment of Alzheimer's disease.

HSPCs, expressing ApoE2, TREM2 and/or metallothioneins are systemically administered to a subject, can migrate to the brain and differentiate into microglia-like cells, thereby replacing the dead or damaged microglial cells. However, as described herein, an alternative process, intracerebroventricular administration of HSPCs, results in faster and more widespread microglia reconstitution. Thus, in some embodiments of the present disclosure, HSPCs are administered to a subject intracerebroventricularly. In some embodiments, the HSPCs are delivered into the cerebrospinal fluid of the cerebral ventricles. This administration route avoids inefficiencies associated with systemically administered compositions having to cross the blood brain barrier. In some embodiments of the present disclosure, intracerebroventricular administration results in faster establishment of progeny cells in the recipient's brain than systemic administration. In some embodiments, the replacement of microglial cells is more widespread using this direct delivery method.

Engraftment and differentiation of HSPCs may be difficult in environments comprising endogenous microglial cells. Endogenous microglial cells may be able to outcompete transplanted HSPCs, and neuroinflammation associated with dying microglial cells may generate an unfavorable environment (e.g., increased inflammation) for HSPC engraftment. To overcome these barriers to HSPC engraftment, in some embodiments, the existing microglial cells are ablated by exposure to an agent capable of removing endogenous microglial cells. For example, pre-transplant administration of a conditioning regimen employing an alkylating agent is an effective means for ablating endogenous microglia precursors (Capotondo et al. (2012); Wilkinson et al. Mol Ther 21, 868-876 (2013), the contents of which are incorporated herein by reference in their entirety). In some embodiments of the present disclosure, the alkylating agent is busulfan. In addition to alkylation agents, such as busulfan, CSF-1R inhibitors (e.g., PLX3397 and PLX5622), and liposomal clodronate may be used (Han et al. Molecular Brain, 10:25, 2017), optionally they may be used in combination with the nanoparticles described, for example, in WO2019191650, which is incorporated herein.

In general, the term nanoparticle refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants. In one embodiment, nanoparticles based on self-assembling bioadhesive polymers are contemplated, which may be applied to oral delivery of agents, intravenous delivery of agents and nasal delivery of agents, all to the brain. Other embodiments, such as oral absorption and ocular deliver of hydrophobic drugs are also contemplated. The molecular envelope technology involves an engineered polymer envelope which is protected and delivered to the site of the disease (Mazza et al. ACS Nano 7, 1016-1026 (2013); Siew et al. Mol Pharm 9, 14-28 (2012); Lalatsa et al. J Control Release 161, 523-536 (2012); Lalatsa et al. Mol Pharm 9, 1665-1680 (2012); Garrett et al. J Biophotonics 5, 458-468 (2012); Uchegbu, Expert Opin Drug Deliv 3, 629-640 (2006); Uchegbu et al. Int J Pharm 224, 185-199 (2001); Qu et al. Biomacromolecules 7, 3452-3459 (2006)).

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation, which includes a particle in accordance with the present invention. A particle in accordance with the present invention is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS).

Particle delivery systems within the scope of the present invention may be provided in any form, including but not limited to solid, semi-solid, emulsion, or colloidal particles. As such any of the delivery systems described herein may be provided as particle delivery systems within the scope of the present invention.

The alkylating agent can be delivered to a subject by any method known in the art. For example, the alkylating agent can be delivered orally, intravenously, intraarterially, intraperitoneally, intramuscularly, subcutaneously, intrathecally, by perfusion through a regional catheter, or by any other means known in the art.

After ablation of endogenous microglial cells or precursors thereof, in some embodiments, HSPCs expressing ApoE2, TREM2 and/or metallothioneins are administered to the subject. In some embodiments, HSPCs are delivered intracerebroventricularly after ablation of the endogenous microglial cells or precursors thereof. In some embodiments, HSPCs are delivered intravascularly after ablation of the endogenous microglial cells or precursors thereof.

### Modified Cells

Some aspects of the present disclosure provide cells that are modified to express one or more exogenous nucleic acid molecules (e.g., ApoE2, TREM2 and/or metallothioneins). In some embodiments, an exogenous nucleic acid molecule encodes a neuroprotective agent (e.g., ApoE2, TREM2 and/or metallothioneins) that ameliorates neuronal loss or dementia in a subject having or suspected of having Alzheimer's disease. In some embodiments, cells are provided that express a neuroprotective agent (e.g., ApoE2, TREM2 and/or metallothioneins) that inhibit neurodegeneration.

The modified cells of the present disclosure are HSPCs in some embodiments. In some embodiments, the cells are microglial cells derived from modified HSPCs. The HSPCs may be isolated from a subject having, suspected of having, or having a propensity to develop Alzheimer's disease. The cells are then modified to express ApoE2, TREM2 and/or metallothioneins, cultured, and administered to the subject. These autologous cells are less likely to elicit an immune response after being administered to the subject than an allogeneic cell. However, in some embodiments, the HSPCs are isolated from a healthy donor. Methods of isolating HSPCs from a donor are known in the art.

### ApoE2

In some embodiments of the present disclosure, the cells of the present disclosure are modified to express Apolipoprotein E (ApoE). In some embodiments of the present disclosure, modified cells express ApoE2 from an exogenous nucleic acid molecule. For example, HSPCs can be modified to express ApoE2 from an exogenous nucleic acid molecule and then administered to a subject, thereby introducing a neuroprotective agent into the brain of a subject having or suspected of having Alzheimer's disease or having a predisposition to develop Alzheimer's disease. In some embodiments, cells derived from the modified HSPCs express ApoE2. For example, in some embodiments, microglia or microglia-like cells express ApoE2 from an exogenous nucleic acid molecule.

### Trem2

Some embodiments of the present disclosure provide modified cells that express Trem2. The modified cells may be HSPCs that are administered to a subject that has or is suspected of having Alzheimer's disease or that has a propensity to develop the disease. In some embodiments, the cells that express Trem2 are cells derived from HSPCs. For example, in some embodiments, modified cells derived from HSPCs are microglia or microglia-like cells.

### Metallothioneins

Some embodiments of the present disclosure provide a modified cell that expresses a metallothionein encoded by an exogenous nucleic acid molecule. The metallothionein is a MT1, MT2, MT3, or MT4 metallothionein in some embodiments. In some embodiments, the metallothionein is a MT1. In some embodiments the MT1 metallothionein is a MT1A, MT1B, MT1E, MT1F, MT1G, MT1H, MT1L, MT1M, or a MT1X metallothionein. In some embodiments, a cell expresses more than one metallothionein from an exogenous nucleic acid molecule. In some embodiments, a cell expresses MT1G from an exogenous nucleic acid molecule. In some embodiments, the cell is an HSPC or its progeny. In some embodiments, the cell is a microglia or microglia-like cell. Metallothioneins are described in International Application Nos. PCT/US2018/013908 and PCT/US2018/013909, the contents of which are incorporated herein by reference in their entirety.

Some embodiments of the present disclosure provide a modified cell that expresses ApoE2, or a fragment thereof, and a metallothionein encoded by exogenous nucleic acid molecules. In some embodiments, the metallothionein is a MT1, MT2, MT3, or MT4 metallothionein. In some embodiments, the modified cell expresses a MT1A, MT1B, MT1E, MT1F, MT1G, MT1H, MT1L, MT1M, or a MT1X metallothionein and ApoE2 or a fragment thereof. In some embodiments, the modified cell expresses MT1G and ApoE2, or a fragment thereof, from exogenous nucleic acid molecules. In some embodiments, the cell is an HSPC or its progeny. In some embodiments, the cell is a microglia or microglia-like cell.

Some embodiments of the present disclosure provide a modified cell that expresses Trem2, or a fragment thereof, and a metallothionein encoded by exogenous nucleic acid molecules. In some embodiments, the metallothionein is a MT1, MT2, MT3, or MT4 metallothionein. In some embodiments, the modified cell expresses a MT1A, MT1B, MT1E, MT1F, MT1G, MT1H, MT1L, MT1M, or a MT1X metallothionein and Trem2 or a fragment thereof In some embodiments, the modified cell expresses MT1G and Trem2, or a fragment thereof, from exogenous nucleic acid molecules. In some embodiments, the cell is an HSPC or its progeny. In some embodiments, the cell is a microglia or microglia-like cell.

Some embodiments of the present disclosure provide a modified cell that expresses ApoE2, Trem2, or a fragment thereof, and a metallothionein encoded by exogenous nucleic acid molecules. In some embodiments, the metallothionein is a MT1, MT2, MT3, or MT4 metallothionein. In some embodiments, the modified cell expresses a MT1A, MT1B, MT1E, MT1F, MT1G, MT1H, MT1L, MT1M, or a MT1X metallothionein, ApoE2, and Trem2 or a fragment thereof. In some embodiments, the modified cell expresses MT1G, ApoE2, and Trem2, or a fragment thereof, from exogenous nucleic acid molecules. In some embodiments, the cell is an HSPC or its progeny. In some embodiments, the cell is a microglia or microglia-like cell.

### Hemizygous CX3CR1 Cells

CX3CR1, also known as the fractalkine receptor, is a seven-transmembrane domain receptor belonging to G protein-coupled receptors family. Being a G protein-coupled receptor, CX3CR1's role is mostly inhibitory as it acts to reduce production of cAMP and prevent triggering signaling cascades mediated by second messengers. The intracellular pathways controlled by CX3CR1 signaling involve mainly PLC, PI3K, and ERK regulation, which modulate cell migration, adhesion, proliferation and survival. It is expressed in several cell types (e.g., monocytes, natural killer cells, T cells, and smooth muscle cells). Microglia are the only cell in the central nervous system that express CX3CR1, which they express at high levels, particularly during development and in response to brain damage/pathology.

Fractalkine (CX3CL1) is the unique ligand for the chemokine receptor CX3CR1 and is expressed either as membrane-bound molecule or in a soluble form. Fractalkine cleavage is mediated by at least two enzymes, ADAM10 and ADAM17, which are active in homeostatic and inflammatory conditions, respectively. Fractalkine acts mainly as adhesion molecule in its membrane-bound form, while it has chemotactic properties towards CX3CR1 in its soluble form. Local production and membrane expression of CX3CL1 and CX3CR1 are controlled by other cytokines, like TNFα, IL-1, IFNγ, NO, and hypoxia.

Activation of the CX3CR1-CX3CL1 axis leads to maintenance of microglia in a quiescent state and of homeostasis in the neuronal network. Under physiological conditions, CX3CL1 seems to inhibit microglial activation, while in particular conditions a paradoxical promotion of an inflammatory response may occur. Neurons are the greater producers of CX3CL1 in the brain and this axis is important for communication with microglia cells. Astrocytes (GFAP⁺) also display constitutive mRNA expression for CX3CL1. Endothelial cells in the brain and spinal cord, as opposed to those in other locations, do not present constitutive CX3CL1 expression on the surface, which suggests that it is rather dependent on their activation. CX3CL1 and CX3CR1 are also expressed in the choroid plexus.

To enhance the ability of HSPCs to generate microglia-like progeny upon transplantation, a mouse model, hemizygous for CX3CR1, and in which a GFP reporter gene has replaced one CX3CR1 allele (B6.129P-CX3CR1tm1Litt/J) was used to demonstrated that i) transplantation of total bone marrow or HSPCs from donor mice haplo-insufficient for the CX3CR1 gene results in an greater and faster appearance of microglia like donor cells in the recipients' brain, as compared to standard wild type donors, and that ii) in the context of competitive transplantation, haplo-insufficient donor derived cells contribute to a greater extent as compared to wild type donor cells to the repopulation of the hematopoietic organs and brain myeloid compartment of the recipients. A branching study performed on the engrafted cells, showed that CX3CR1^{+/GFP} cells also acquire a more mature microglia-like morphology. CX3CR1 hemizygous mice have no obvious phenotype.

Thus, the present disclosure contemplates isolating HSPCs and knocking out one allele of CX3CR1 to create a hemizygous cell, which can be cultured to generate a population of cells that are hemizygous for CX3CR1. Such cells can be administered to a subject in need thereof. The present disclosure also contemplates modifying a CX3CR1 hemizygous HSPC to incorporate a nucleic acid sequence encoding a ApoE2, TREM2 and/or metallothionein protein in the missing CX3CR1 allele locus. In this way, the hemizygous HSPCs are manipulated to express a therapeutic agent. Alternatively, an isolated HSPC may be edited to remove one copy of CX3CR1 to generate a hemizygous HSPC. Editing a single copy of the CX3CR1 comprises, in some embodiments, replacing the CX3CR1 allele with an exogenous nucleic acid molecule encoding a therapeutic agent. Such editing is carried out using any method known in the art.

### Modifying Cells

To produce any of the CX3CR1 hemizygous cells described herein, any approach known in the art may be used. For example, cells may be modified by editing endogenous genes, such as CX3CR1. Gene editing is a major focus of biomedical research, embracing the interface between basic and clinical science. “Gene editing” tools can manipulate a cell's DNA sequence at a specific chromosomal locus without introducing mutations at other sites of the genome. This technology effectively enables a researcher to manipulate the genome of a cell in vitro or in vivo.

In one embodiment, gene editing involves targeting an endonuclease to a specific site in a genome to generate a double strand break at the specific location. If a donor DNA molecule (e.g., a plasmid or oligonucleotide) is introduced, interactions between the nucleic acid comprising the double strand break and the introduced DNA can occur, especially if the two nucleic acids share homologous sequences. In this instance, a process termed “gene targeting” can occur, in which the DNA ends of the chromosome invade homologous sequences of the donor DNA by homologous recombination. By using the donor plasmid sequence as a template for homologous recombination, a seamless knock out of the gene of interest can be accomplished. Importantly, if the donor DNA molecule includes a deletion within the target gene (e.g., CX3CR1), homologous recombination-mediated double strand break repair will introduce the donor sequence into the chromosome, resulting in the deletion being introduced within the chromosomal locus. By targeting the nuclease to a genomic site that contains the target gene, the concept is to use double strand break formation to stimulate homologous recombination and to thereby replace the functional target gene with a deleted form of the gene. The advantage of the homologous recombination pathway is that it has the potential to generate seamlessly a knockout of the gene in place of the previous wild-type allele.

Genome editing tools may use double strand breaks to enhance gene manipulation of cells. Such methods can employ zinc finger nucleases, described for example in U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; and 6,479,626; and U.S. Pat. Publ. Nos. 20030232410 and US2009020314, which are incorporated herein by reference); Transcription Activator-Like Effector Nucleases (TALENs; described for example in U.S. Pat. Nos. 8,440,431; 8,440,432; 8,450,471; 8,586,363; and 8,697,853; and U.S. Pat. Publ. Nos. 20110145940; 20120178131; 20120178169; 20120214228; 20130122581; 20140335592; and 20140335618; which are incorporated herein by reference), and the CRISPR (Clustered Regularly Interspaced Short Palindromic Repeats)/Cas9 system (described for example in U.S. Pat. Nos. 8,697,359; 8,771,945; 8,795,965; 8,871,445; 8,889,356; 8,906,616; 8,932,814; 8,945,839; 8,993,233; and 8,999,641; and U.S. Pat. Publ. Nos. 20140170753; 20140227787; 20140179006; 20140189896; 20140273231; 20140242664; 20140273232; 20150184139; 20150203872; 20150031134; 20150079681; 20150232882; and 20150247150, which are incorporated herein by reference). For example, zinc finger nuclease DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. Zinc finger nucleases and TALENs entail use of modular sequence-specific DNA binding proteins to generate specificity for about 18 bases sequences in the genome.

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break. The zinc finger nuclease is then repaired either by non-homologous end joining, which leads to insertion/deletion (indel) mutations, or by homology-directed repair, which requires an exogenous template and can generate a precise modification at a target locus (Mali et al., Science, Feb. 15, 2013; 339 (6121): 823-6, the contents of which are herein by reference in their entirety). Unlike gene therapy methods that add a functional, or partially functional, copy of a gene to a subject's cells but retain the original dysfunctional copy of the gene, this system can remove the defect in the dysfunctional copy. Genetic correction using modified nucleases has been demonstrated in tissue culture cells and rodent models of rare diseases.

CRISPR has been used in a wide range of organisms including baker's yeast (S. cerevisiae), zebra fish, nematodes (e.g., C. elegans), plants, mice, and several other organisms.

Additionally, CRISPR has been modified to make programmable transcription factors that allow scientists to target and activate or silence specific genes. Libraries of tens of thousands of guide RNAs are now available. By inserting a plasmid containing cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length, with some CRISPR spacer sequences exactly matching sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define eight CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about thirty base pairs in length), which are then inserted into the CRISPR locus near the leader sequence. RNAs from the CRISPR loci are constitutively expressed and are processed by Cas proteins to small RNAs comprising individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype Ecoli) proteins (called CasA-E in E. coli) form a functional complex, Cascade, that processes CRISPR RNA transcripts into spacer-repeat units that Cascade retains. In other prokaryotes, Cas6 processes CRISPR transcripts. Interestingly, CRISPR-based phage inactivation in E. coli requires Cascade and Cas3, but neither Cas1 nor Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

As an RNA guided protein, Cas9 requires an RNA molecule to direct the recognition of DNA targets. Though Cas9 preferentially interrogates DNA sequences containing a protospacer adjacent motif (PAM) sequence (i.e., NGG). However, the Cas9-gRNA complex requires a substantial complementarity between the guide RNA (gRNA) and the target nucleic acid sequence to create a double strand break. Synthetic gRNA can be designed to combine the essential RNA sequences for Cas9 targeting into a single RNA expressed with the RNA polymerase type 21 promoter U6 driving expression. Synthetic gRNAs are slightly over 100 bases at the minimum length and contain a portion which is targets the 20 protospacer nucleotides immediately preceding the PAM sequence NGG.

In one approach, an HSPC cell is altered to delete or inactivate a CX3CR1 allele using a CRISPR-Cas system. Cas9 can be used to target a CX3CR1 gene. Upon target recognition, Cas9 induces double strand breaks in the CX3CR1 target gene. Homology-directed repair (HDR) at the double-strand break site can allow insertion of an inactive or deleted form of the CX3CR1 sequence. In some embodiments, homology-directed repair (HDR) at the double-strand break site can allow insertion of an expression cassette of the invention.

In one approach, an HSPC cell is altered to delete or inactivate a TSPO allele using a CRISPR-Cas system. Cas9 can be used to target a TSPO gene. Upon target recognition, Cas9 induces double strand breaks in the TSPO target gene. Homology-directed repair (HDR) at the double-strand break site can allow insertion of an inactive or deleted form of the TSPO sequence. In some embodiments, homology-directed repair (HDR) at the double-strand break site can allow insertion of an expression cassette of the invention.

The following US patents and patent publications are incorporated herein by reference: U.S. Pat. No. 8,697,35; 20140170753; 20140179006; 20140179770; 20140186843; 20140186958; 20140189896; 20140227787; 20140242664; 20140248702; 20140256046; 20140273230; 20140273233; 20140273234; 20140295556; 20140295557; 20140310830; 20140356956; 20140356959; 20140357530; 20150020223; 20150031132; 20150031133; 20150031134; 20150044191; 20150044192; 20150045546; 20150050699; 20150056705; 20150071898; 20150071899; 20150071903; 20150079681; 20150159172; 20150165054; 20150166980; and 20150184139.

### Expression of Exogenous Therapeutic Agents

ApoE2, TREM2 and/or Metallothioneins are therapeutic polypeptides useful for the treatment of Alzheimer's diseases. Polynucleotide encoding such proteins are inserted into expression vectors by techniques known in the art. For example, double stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

The present disclosure also includes vectors (e.g., recombinant plasmids) that include nucleic acid molecules encoding ApoE2, TREM2 and/or Metallothioneins as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector may include a nucleotide sequence encoding a polypeptide, or fragment thereof, operatively linked to regulatory sequences such as promoter sequences, terminator sequences, long terminal repeats, untranslated regions, enhancers, and the like, as defined herein. Recombinant expression vectors allow for expression of the genes or nucleic acids included in them. In particular embodiments, a promoter is described in U.S. Provisional Application No. 62/908,966, which is incorporated by reference in its entirety. In some embodiments, the promoter comprises a translocator protein (TPSO) promoter. In some embodiments, the regulatory sequences comprise a translocator protein (TPSO) promoter in combination with one or more enhancers. In some embodiments, the regulatory sequences comprise P1, P2, P1+P2, or P2+P1 alone or in combination with one or more of E1, E2, E1.1, and E1.2. In some embodiments, the regulatory sequences comprises P1+P2, P1, or E1+P1, where the sequence to the left of “+” is oriented 5′ to the sequence right of the “+”.

In some embodiments of the present disclosure, one or more DNA molecule having a nucleotide sequence encoding one or more polypeptides or polynucleotides described herein are operatively linked to one or more regulatory sequences, which can integrate the desired DNA molecule into a eukaryotic cell. Cells (e.g., HSPCs, microglia) which have been stably transfected or transduced by the introduced DNA can be selected, for example, by introducing one or more markers which allow for selection of host cells which contain the expression vector. A selectable marker gene can either be linked directly to a nucleic acid sequence to be expressed, or be introduced into the same cell by co-transfection or co-transduction. Any additional elements needed for optimal synthesis of polynucleotides or polypeptides described herein would be apparent to one of ordinary skill in the art.

In some embodiments, an HSPC may be modified by introducing an exogenous nucleic acid molecule into the cell. The exogenous nucleic acid may comprise a transgene encoding a therapeutic agent for the treatment of Alzheimer's disease. The exogenous nucleic acid, in some embodiments, comprises regulatory elements for expressing a transgene. For example, an exogenous nucleic acid molecule may comprise a transgene encoding a therapeutic agent for the treatment of Alzheimer's disease and a promoter for expressing the transgene. In some embodiments, the promoter is a constitutively active promoter such as, for example, the cytomegalovirus (CMV), simian virus 40 (SV40) promoter. In some embodiments, the promoter may be a tissue-specific promoter, wherein the transgene is expressed upon engraftment and differentiation of the HSPC. For example, tetracycline is a drug that can be used to activate a tetracyclin-sensitive promoter. In some embodiments, a neuronal specific promoter is the synapsin (Syn) promoter. In some embodiments, the promoter may be an inducible promoter, wherein the transgene is expressed only in the presence or absence of a particular compound. In some embodiments, microglial or microglial-like cells derived from an HSPC comprising a transgene driven by a brain-specific promoter transplanted into the brain of a subject will express the transgene. In some embodiments, the exogenous nucleic acid molecule may comprise, in addition to a transgene, a detectable label or other marker that allows identification of cells that have been successfully modified or that are derived from cells that have been successfully modified to express the transgene.

Methods of introducing exogenous nucleic acid molecules into a cell are known in the art. For example, eukaryotic cells can take up nucleic acid molecules from the environment via transfection (e.g., calcium phosphate-mediated transfection). Transfection does not employ a virus or viral vector for introducing the exogenous nucleic acid into the recipient cell. Stable transfection of a eukaryotic cell comprises integration into the recipient cell's genome of the transfected nucleic acid, which can then be inherited by the recipient cell's progeny.

Eukaryotic cells (i.e., HSPCs) can be modified via transduction, in which a virus or viral vector stably introduces an exogenous nucleic acid molecule to the recipient cell. Eukaryotic transduction delivery systems are known in the art. Transduction of most cell types can be accomplished with retroviral, lentiviral, adenoviral, adeno-associated, and avian virus systems, and such systems are well-known in the art. While retroviruses systems are generally not compatible with neuronal cell transduction, lentiviruses are a genus of retroviruses well-suited for transducing stem cells as well as neuronal cells. Thus, in some embodiments of the present disclosure, the viral vector system is a lentiviral system. In some embodiments, the viral vector system is an avian virus system, for example, the avian viral vector system described in U.S. Pat. No. 8,642,570, DE102009021592, PCT/EP2010/056757, and EP2430167, the contents of which are incorporated herein by reference in their entirety. In some embodiments, the viral vectors are assembled or packaged in a packaging cell prior to contacting the intended recipient cell. In some embodiments, the vector system is a self-inactivating system, wherein the viral vector is assembled in a packaging cell, but after contacting the recipient cell, the viral vector is not able to be produced in the recipient cell.

The components of a viral vector are encoded on plasmids, and because efficiencies of transduction decrease with large plasmid size, multiple plasmids that have different viral sequences necessary for packaging may be necessary. For example, in a lentiviral vector system, a first plasmid may comprise a nucleotide sequence encoding a Group antigens (gag) and/or a reverse transcriptase (pol) gene, while a second plasmid encodes regulator of expression of virion proteins (rev) and/or envelope (env) genes. The exogenous nucleic acid molecule comprising a transgene can be packaged into the vector and delivered into a recipient cells where the transgene is integrated into the recipient cell's genome. Additionally, the transgene may be packaged using a split-packaging system as described in U.S. Pat. No. 8,642,570, DE102009021592, PCT/EP2010/056757, and EP2430167.

After the introduction of one or more vector(s), host cells are cultured prior to administration to a subject. In some embodiments, the Expression of recombinant proteins can be detected by immunoassays including Western blot analysis, immunoblot, and immunofluorescence. Purification of recombinant proteins can be carried out by any of the methods known in the art or described herein, for example, any conventional procedures involving extraction, precipitation, chromatography, and electrophoresis. A further purification procedure that may be used for purifying proteins is affinity chromatography using monoclonal antibodies which bind a target protein. Generally, crude preparations containing a recombinant protein are passed through a column on which a suitable monoclonal antibody is immobilized. The protein usually binds to the column via the specific antibody while the impurities pass through. After washing the column, the protein is eluted from the gel by changing pH or ionic strength, for example.

### Pharmaceutical Compositions

Compositions contemplated in the present disclosure include pharmaceutical compositions comprising cells expressing a neuroprotective agent. In some embodiments, the neuroprotective agent is ApoE2, Trem2, and/or metallothionein. Pharmaceutical compositions can comprise autogenic or allogenic cells that are modified to express a therapeutic agent.

Hematopoietic stem progenitor cells (HSPCs) as described herein can be administered as therapeutic compositions (e.g., as pharmaceutical compositions). Cellular compositions as described herein can be provided as sterile liquid preparations, e.g., isotonic aqueous solutions, suspensions, emulsions, dispersions, or viscous compositions, which may be buffered to a selected pH. A liquid preparation may be easier to prepare than a gel, another viscous composition, and a solid composition. Additionally, a liquid composition may be more convenient to administer (i.e., by injection). Viscous compositions, on the other hand, can be formulated within the appropriate viscosity range to provide longer contact periods with specific tissues. Liquid or viscous compositions can comprise a carrier, which can be a solvent or dispersing medium comprising, for example, water, saline, phosphate buffered saline, polyol (e.g., glycerol, propylene glycol, liquid polyethylene glycol, and the like), and suitable mixtures thereof.

Sterile injectable solutions can be prepared by incorporating the cells described herein in a sufficient amount of an appropriate diluent. Such compositions may be in admixture with a suitable carrier or excipient such as sterile water, physiological saline, glucose, dextrose, or another carrier or excipient suitable for delivering live cells to a subject. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “Remington's Pharmaceutical Science”, 17th edition, 1985, incorporated herein by reference, may be consulted to prepare suitable preparations, without undue experimentation.

Additives that enhance the stability and sterility of the cellular compositions, including antimicrobial preservatives, antioxidants, chelating agents, and buffers, can be added. Prevention of the action of microorganisms can be ensured by an antibacterial or antifungal agent including, but not limited to, parabens, chlorobutanol, phenol, and sorbic acid. According to the present disclosure, however, any vehicle, diluent, or additive used must be compatible with the cells.

The compositions can be isotonic, i.e., they have the same osmotic pressure as blood and cerebrospinal fluid. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride may be suitable for buffers containing sodium ions.

Viscosity of the compositions, if desired, can be maintained at a selected level using a pharmaceutically acceptable thickening agent. In some embodiments, the thickening agent is methylcellulose, which is readily and economically available and is easy to work with. Other suitable thickening agents include, but are not limited to, xanthan gum, carboxymethyl cellulose, hydroxypropyl cellulose, and carbomer. The concentration of the thickener will depend upon the agent selected and the amount of the agent used. Suitable carriers and other additives may be chosen depending on the route of administration and the nature of the dosage form (e.g., a liquid dosage form can be formulated into a solution, a suspension, a gel, or another liquid form, such as a time release formulation or liquid-filled form).

An effective amount of cells to be administered can vary for the subject being treated. In one embodiment, between about 10⁴ to about 10⁸ cells, and in another embodiment between about 10⁵ to about 10⁷ cells are administered to a subject.

The skilled artisan can readily determine the amounts of cells and optional additives, vehicles, and/or carrier in compositions to be administered. In one embodiment any additive (in addition to the cell(s)) is present in an amount of about 0.001% to about 50% (weight) solution in phosphate buffered saline, and the active ingredient is present in the order of micrograms to milligrams, such as about 0.0001% to about 5 wt %. In another embodiment, the active ingredient is present at about 0.0001% to about 1 wt %. In yet another embodiment, the active ingredient is present at about 0.0001% to about 0.05 wt %. In still other embodiments, the active ingredient is present at about 0.001% to about 20 wt %. In some embodiments, the active ingredient is present at about 0.01% to about 10 wt %. In another embodiment, the active ingredient is present at about 0.05% to about 5 wt %. For any composition to be administered to an animal or human, and for any particular method of administration, toxicity can be determined by measuring the lethal dose (LD) and LD₅₀ in a suitable animal model e.g., a rodent such as mouse. The dosage of the composition(s), concentration of components therein, and timing of administering the composition(s), which elicit a suitable response can also be determined. Such determinations do not require undue experimentation in light of the knowledge of the skilled artisan, this disclosure, and the documents cited herein. The time for sequential administrations can also be ascertained without undue experimentation.

### Methods of Treatment

A health care professional may diagnose a subject as having a neurodegenerative disease by the assessment of one or more symptoms of a neurodegenerative disease in the subject. Non-limiting symptoms of a neurodegenerative disease in a subject include difficulty lifting the front part of the foot and toes; weakness in arms, legs, feet, or ankles; hand weakness or clumsiness; slurring of speech; difficulty swallowing; muscle cramps; twitching in arms, shoulders, and tongue; difficulty chewing; difficulty breathing; muscle paralysis; partial or complete loss of vision; double vision; tingling or pain in parts of body; electric shock sensations that occur with head movements; tremor; unsteady gait; fatigue; dizziness; loss of memory; disorientation; misinterpretation of spatial relationships; difficulty reading or writing; difficulty concentrating and thinking; difficulty making judgments and decisions; difficulty planning and performing familiar tasks; depression; anxiety; social withdrawal; mood swings; irritability; aggressiveness; changes in sleeping habits; wandering; dementia; loss of automatic movements; impaired posture and balance; rigid muscles; bradykinesia; slow or abnormal eye movements; involuntary jerking or writhing movements (chorea); involuntary, sustained contracture of muscles (dystonia); lack of flexibility; lack of impulse control; and changes in appetite. A health care professional may also base a diagnosis, in part, on the subject's family history of a neurodegenerative disease. A health care professional may diagnose a subject as having a neurodegenerative disease upon presentation of a subject to a health care facility (e.g., a clinic or a hospital). In some instances, a health care professional may diagnose a subject as having a neurodegenerative disease while the subject is admitted in an assisted care facility. Typically, a physician diagnoses a neurodegenerative disease in a subject after the presentation of one or more symptoms.

The present disclosure provides methods of treating Alzheimer's disease or symptoms thereof which comprise administering to a subject (e.g., a mammal, such as a human) a therapeutically effective amount of a pharmaceutical composition comprising a cell expressing a neuroprotective agent, such as ApoE2, Trem2, MT1G, or a combination thereof. In some embodiments, the cell is a hematopoietic stem progenitor cell. In some embodiments, the cell is a microglial progenitor cell. Thus, the method in some embodiments comprises administering to the subject a therapeutically effective amount of a cell described herein sufficient to treat Alzheimer's disease or symptom thereof, under such conditions that Alzheimer's disease is treated.

The methods herein include administering to the subject (including a subject identified as in need of such treatment) an effective amount of cells described herein, or a composition comprising such cells as described herein to produce such effect. Such treatment will be suitably administered to a subject, particularly a human, suffering from, having, susceptible to, or at risk for, Alzheimer's disease, or a symptom thereof. In some embodiments, the methods herein include administering to the subject (including a subject identified as in need of such treatment) an effective amount of a compound described herein, or a composition described herein to produce such effect.

In some embodiments, the cell or the composition comprising the cell is administered to a subject in a targeted manner. For example, in some embodiments, a composition comprising a cell expressing ApoE2, Trem2, or a metalloprotein, or a combination thereof, is administered directly to a subject's brain. In some embodiments, the composition is delivered directly to the brain via intracerebroventricular administration. In some embodiments, the composition is delivered in this manner to the lateral ventricles of the subject's brain. Methods of administration useful in the present disclosure are described, for example in International Application No. PCT/US2020/045106, which is incorporated by reference in its entirety.

Alternatively, the composition may be delivered systemically, such as by intravenous administration. Cells administered in such a manner must traverse the blood brain barrier prior to engrafting in the subject's brain. Other modes of administration (parenteral, mucosal, implant, intraperitoneal, intradermal, transdermal, intramuscular, intracerebroventricular injection, intravenous including infusion and/or bolus injection, and subcutaneous) are generally known in the art. In some embodiments, cells are administered in a medium suitable for injection, such as phosphate buffered saline, into a subject. Because the cells being administered to the subject are intended to repopulate microglial cells, intracerebroventricular administration may be advantageous as other routes of administration require crossing the blood brain barrier.

Engraftment of transplanted cells into a subject's brain provides a population of cells that express a therapeutic agent. But because the transplanted cells are meant to replace endogenous cells (i.e., microglial cells), in certain embodiments, methods of treating a subject having, susceptible to, or at risk of developing Alzheimer's disease further comprise administering to a subject prior to administering an HSPC expressing a therapeutic agent, an agent for ablating endogenous microglia. In some embodiments, the agent is an alkylating agent. In particular, nanoparticle delivery of alkylating agents may be effective in creating a suitable environment for engraftment of transplanted HSPCs, as described in International Application No. PCT/US2017/056774, the contents of which are incorporated herein by reference in their entirety.

### Kits

The present disclosure contemplates kits for the treatment or prevention of Alzheimer's disease. In some embodiments, the kit comprises a composition comprising a modified HSPC expressing a neuroprotective agent. In some embodiments, the neuroprotective protein is an ApoE2, Trem2, and a metallothionein, or a combination thereof. The kit can include instructions for a treatment protocol, reagents, equipment (test tubes, reaction vessels, needles, syringes, etc.), and standards for calibrating or conducting the treatment protocol. The instructions provided in a kit according to the present disclosure may be directed to suitable operational parameters in the form of a detectable label or a separate insert. Optionally, the kit may further comprise a standard or control information so that the test sample can be compared with the control information standard to determine if a consistent result is achieved. In some embodiments, the kit includes a nanoparticle for ablative conditioning of endogenous microglial cells.

In some embodiments, the kit comprises a sterile container which contains a therapeutic or prophylactic cellular composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing a neurological disease or disorder of the central nervous system. The instructions will generally include information about the use of the composition for the treatment or prevention of the disease or disorder. In other embodiments, the instructions include at least one of the following: description of the therapeutic agent; dosage schedule and administration for treatment or prevention of a neurological disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples provide those of ordinary skill in the art with a complete description of how to make and use the compositions and therapeutic methods of the disclosure, and are not intended to limit the scope of what the inventors regard as their invention.

### EXAMPLES

### Example 1: Lentiviral Vector Generation and Characterization

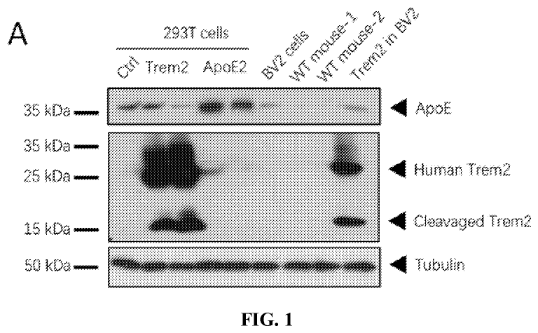

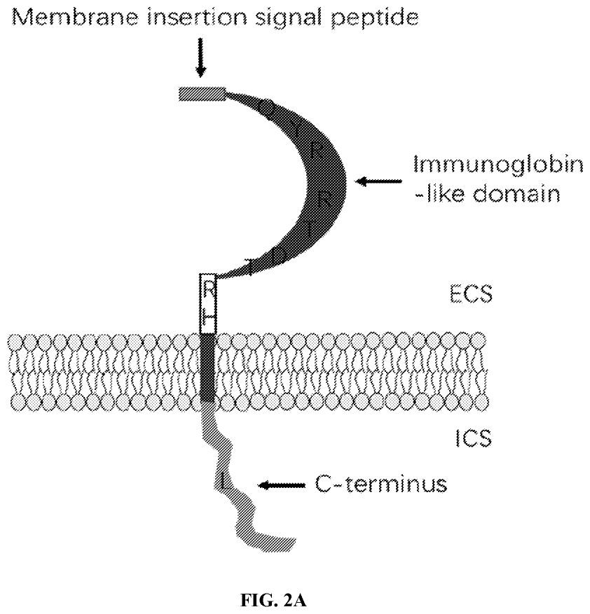

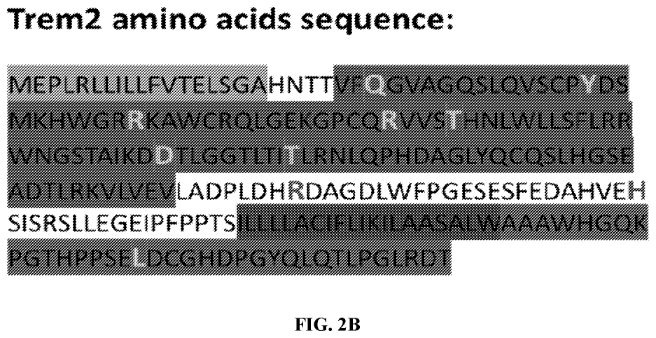

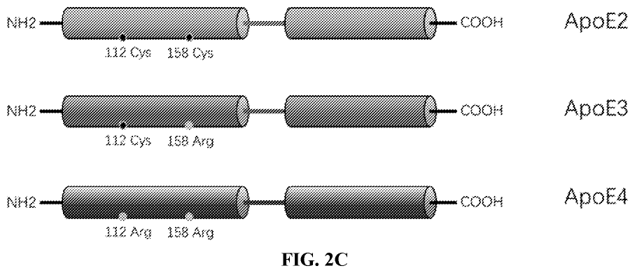

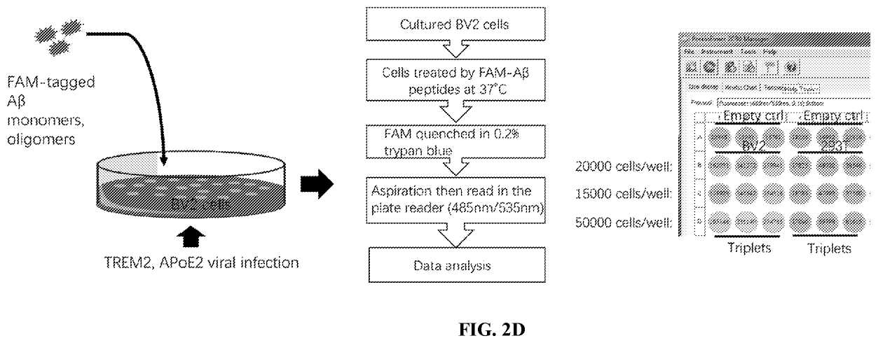

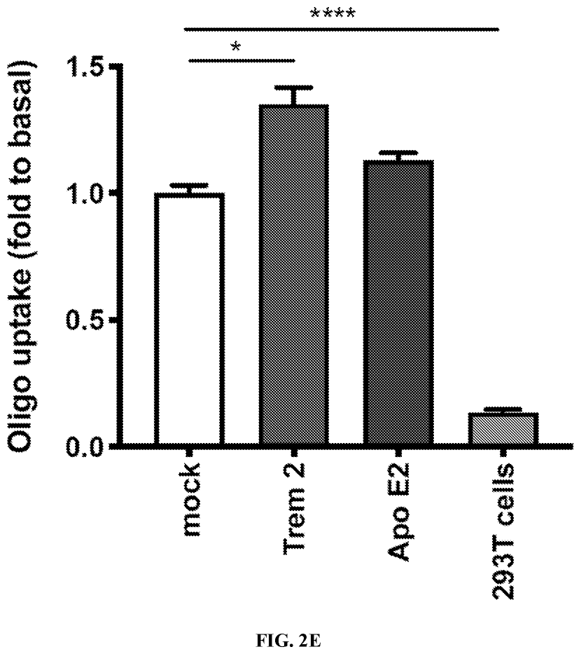

Lentiviral vectors comprising nucleic acids encoding human ApoE2 (hApoE2) and Trem2 (hTrem2) under the transcriptional control of the human PGK promoter were generated and tested for expression of the target molecules in BV-2 cells (FIG. 1 and FIGS. 2A to 2C). Trem2 is an immune receptor in the brain that is expressed on the cell membrane so that its N-terminus is exposed to the extracellular space.

The Trem2 and ApoE2 vectors were employed for an in vitro experiment on BV-2 microglia cells. Correct cell surface expression was confirmed by flow cytometry on Trem2-transduced BV-2 cells (not shown).

The capability of Amyloid β (Aβ) engulfment by mock treated and Trem2_LV and ApoE2-LV transduced BV2 cells was assessed with a fluorescent imaging system. Properly processed 5-FAM tagged Amyloid β (Aβ) monomer (150 in 1×PBS) and oligomers (150 in 1×PBS, incubated at 37° C. for 72 hrs) were used to treat stably transduced and control BV2 cells then read with a fluorescent microplate reading system (FIG. 2D). As expected, BV-2 microglia engulfed abundantly Amyloid β (Aβ) oligomers in comparison to control 293T cells (FIG. 2E), Interestingly, Amyloid f3 (Aβ) oligomer engulfment was increased in Trem2 transduced BV-2 cells as compared to control untransduced BV-2 (FIG. 2E).

### Example 2: Hematopoietic Stem Cell Gene Therapy in 5×FAD Mice

An experiment was designed to explore the use of hematopoietic stem cell (HSC) gene therapy with ApoE2 and Trem2+/−metallothionein (MT) overexpressing lentiviral vectors (LVs) in a reliable animal model of Alzheimer's disease (AD), namely 5×FAD mice. As Alzheimer's disease (AD) only affects the central nervous system (CNS), an innovative transplant protocol was employed based on transduced hematopoietic stem progenitor cell (HSPC) delivery into the brain lateral ventricles of recipient mice, associated to an untransduced hematopoietic cell back up for rescue from busulfan myeloablative conditioning. The overall purpose of this study was to assess the efficacy of an intra-brain gene therapy approach based on the transplantation of murine hematopoietic stem progenitor cells (HSPCs) transduced with lentiviral vectors (LVs) encoding the cDNA for human ApoE2 or Trem2 genes in combination with metallothioneins (MTs), and select the most promising transcripts for future development.

Numerous transgenic mouse models of Alzheimer's disease (AD) were generated over past few years to characterize underlying pathological and behavioral alterations. 5×FAD transgenic mice possess 5 mutations and these mutations lead to overproduction of Aβ42 and the mice exhibit amyloid plaque pathology similar to that found in Alzheimer's disease (AD), while other transgenic models develop plaques slowly. Intra-neuronal Aβ42 accumulation in 5λFAD brain starts at the age of 6 weeks. These mice develop early onset of AD disease at approximately 6 weeks of age. The survival of 5×FAD mice has been reported to be decreased after 10 months.

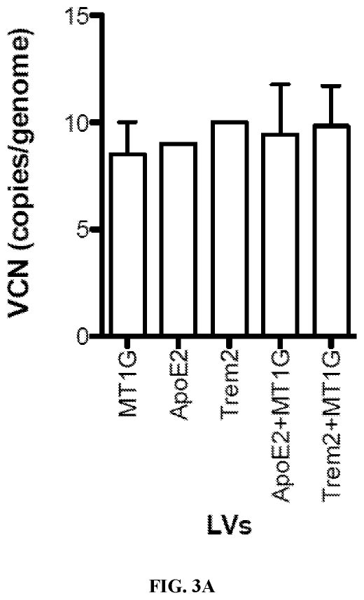

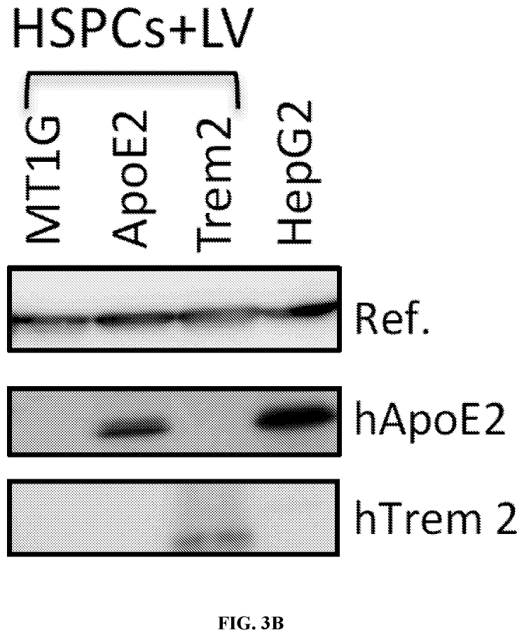

Lineage negative cells (Lin−) were purified from the bone marrow of donor mice (5×FAD) and then transduced with one of the following vectors: APOE2/TREM2 or their combination with MT1G according to standardized transduction protocols at multiplicity of infection (MOI) 75-100. Good and comparable transduction efficiency was obtained in the preparation of all samples in which a vector copy number of about 10 was measured on in vitro liquid culture (FIG. 3A). Expression of hApoE2 and Trem2 was confirmed in the liquid culture progeny of the transduced cell by Western blot (FIG. 3B).

These transduced cells (0.3×10e6 to 0.5×10e6 lentiviral vector- (LV) transduced Lin-cells) were injected into the brain of the Busulfan myeloablated 5×FAD recipient mice through intra-cerebral ventricular (ICV) injection. Bone marrow support was given to rescue peripheral hematopoiesis (2.0×10e6 total bone marrow cells which may or may not be transduced with lentiviral vector (LV) containing reporter gene like green fluorescent protein (GFP) or left not transduced) 3-5 days post transplantation. Between 10 and 15 animals were included in each treatment cohort. Controls included 5×FAD mice left untreated or transplanted with untransduced/GFP-transduced 5×FAD lin− cells, and wild type animals left untreated or transplanted with un-transduced/GFP-transduced wild type lin− cells, all age matched to treated animals. Diazepam was administered for 9 days, in the drinking water as Seizures prophylaxis. Baytril for two weeks followed by Sulfatrim was given in oral solution subsequently as antibiotic prophylaxis. After transplant mice were monitored with collection of clinical signs for early identification of inter-current deaths (ICDs) until scheduled terminal assessments.

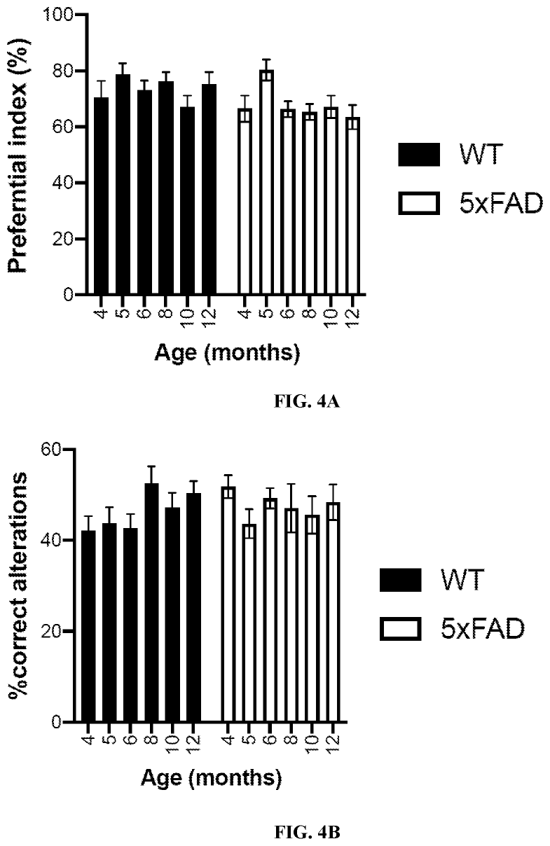

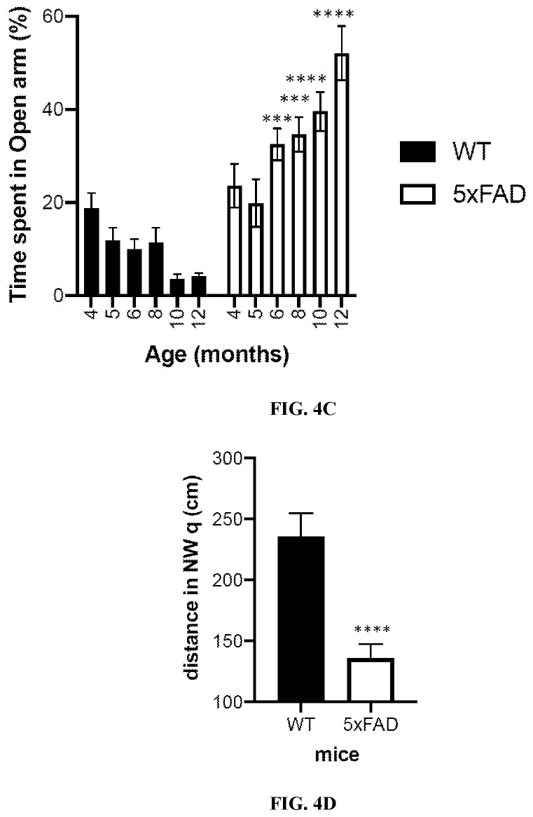

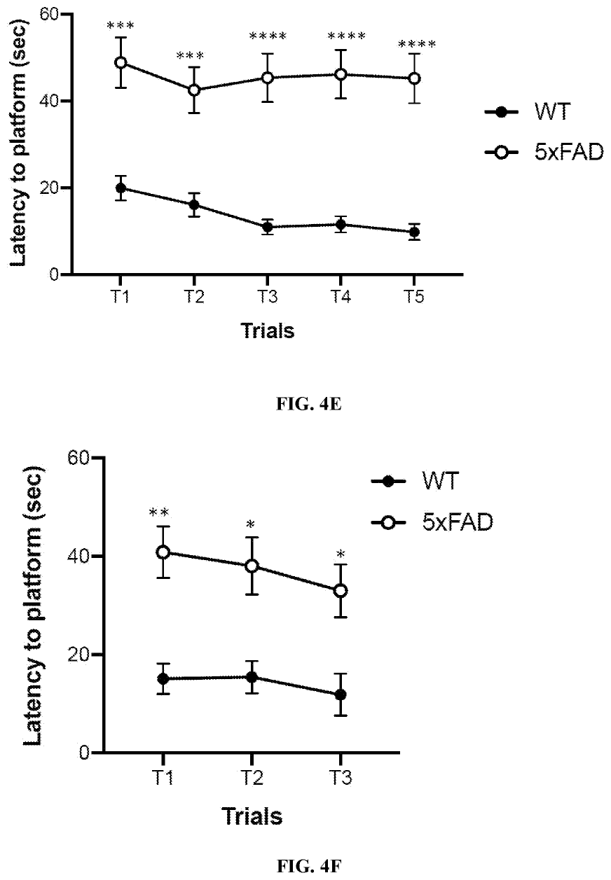

The 5×FAD mouse model developed an age-dependent motor phenotype in addition to working memory deficits in an alternation task and reduced anxiety levels as revealed in the elevated plus maze task. For phenotypic assessment, behavioral tests were performed every month from age 4 months until the age of 12 months (Novel object recognition, Open field test, and Elevated plus maze) for phenotype variation monitoring and assessment of beneficial effect of gene therapy in the transplanted mice, except Morris Water Maze (MWM) test which was done to document cognitive deficits at the age of 12 months when the endpoint of study was performed. Among the tests performed, the Elevated plus maze demonstrated a clear phenotypic progression of Alzheimer's disease (AD) in control animals (green fluorescent protein- (GFP) transplanted and untreated 5×FAD mice), while the other tests (Novel object recognition, Open field test) were not informative in the experimental setting (FIGS. 4A to 4F). The Morris water maze (MWM) revealed a pathologic phenotype as reduced ability to reach a hidden and reverse platform over multiple trials and little distance travelled in the target quadrant (NW) (probe trial) (FIGS. 4A to 4F).

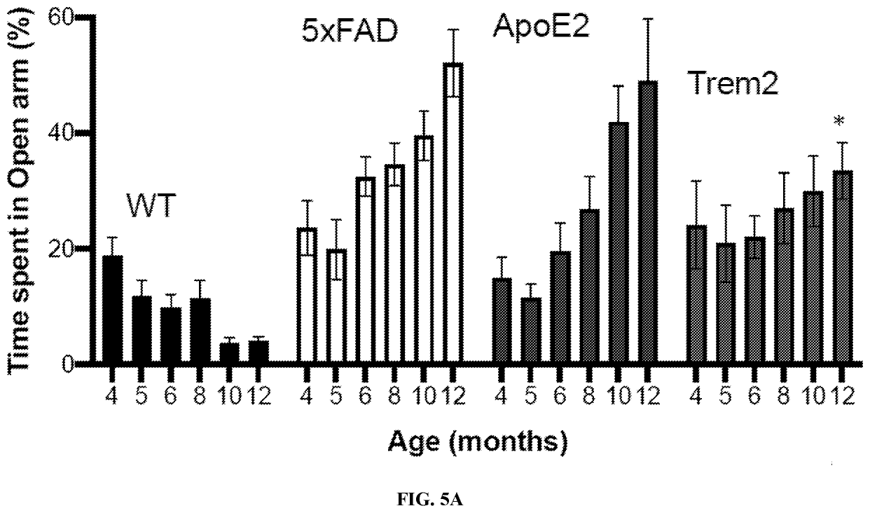

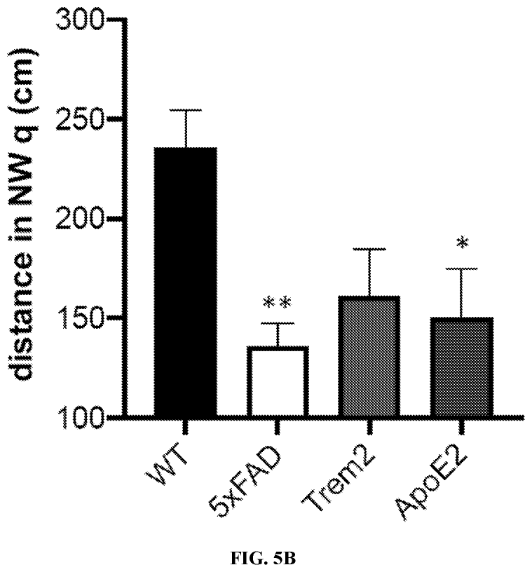

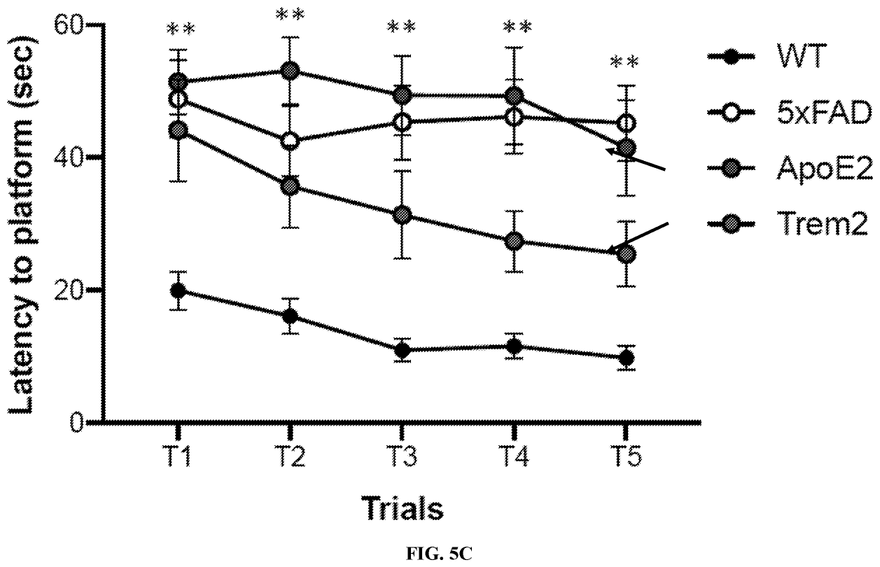

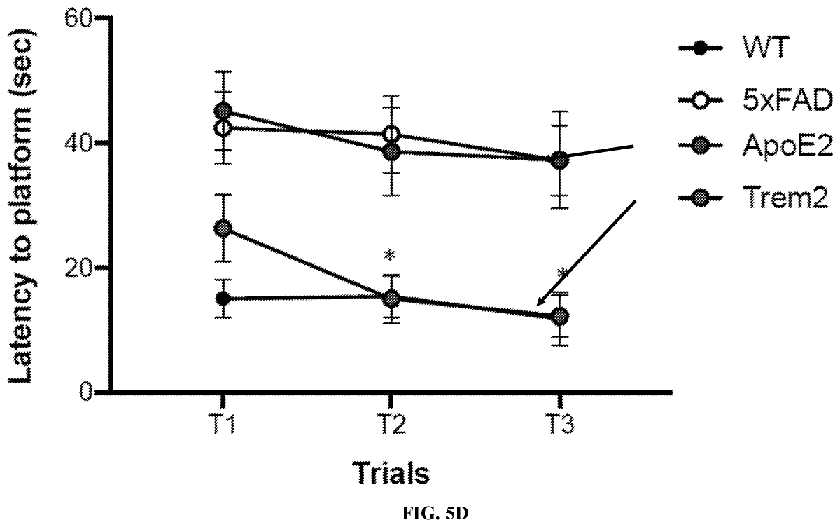

These tests were also employed to evaluate treated mice phenotype. Interestingly, the elevated plus maze for the assessment of anxiety showed less time spent in open arm in the in Trem2-transplanted animals vs. control affected mice at the 12 months-time point, with a prevention of the pathologic phenotype observed in the controls of the same age (FIG. 5A). Similar results were observed at the Morris water maze (MWM) test (FIG. 5B-D). In the probe trial, all 5×FAD control mice showed a significant decrease in the distance travelled in the target quadrant (NW) while wild type and Trem2-treated animals covered more distance, indicating they had improved memory retention or recall as compared to 5×FAD controls. Moreover, Trem2-transplanted animals showed a significant improvement in the latency to find the hidden platform after 5 trials. Furthermore, the deficit of cognitive flexibility observed in 5×FAD during reversal learning in the Morris water maze (MWM) task was prevented by Trem2-transduced cell transplantation. Indeed, the average latency to reach the platform was significantly lower in Trem2-treated animals than in 5×FAD control mice, suggesting better cognitive flexibility as good as that observed in wild type animals. ApoE2-treated animals instead did not significantly differ from 5×FAD controls at behavioral testing. Transduction of the transplanted cells with the MT1G lentiviral vector (LV) in addition to the vector encoding for Trem2 or ApoE2 did not affect the outcome of the behavioral tests in the respective cohorts (data not shown).

After behavioral testing, the treated and control animals underwent terminal evaluation, inclusive of full necropsy, after trans-cardiac perfusion with saline and tissue collection for molecular and histological evaluations. Digital droplet PCR (ddPCR) performed on the brain of transplanted animals documented the engraftment of the transduced cells (whose vector copy number (VCN) measured on the liquid culture progeny is shown in FIG. 3A) with vector copy number (VCN) values ranging between 0.5 and 1.5 copies of LV/murine genome, with the highest vector copy number (VCN) measured in the APoE2+MT1G cohort. ddPCR failed to reveal engraftment of the transduced cells in the bone marrow (not shown).

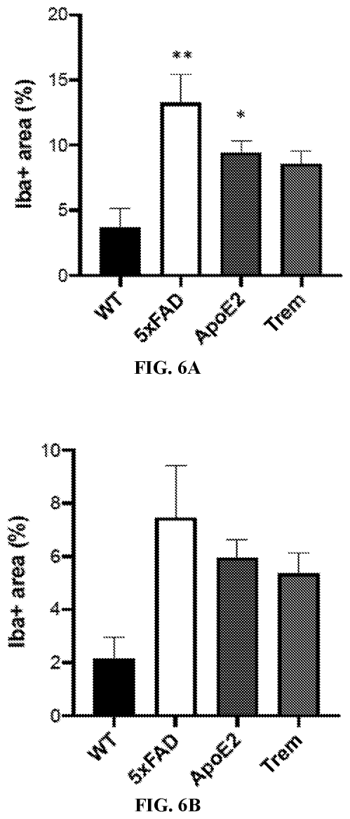

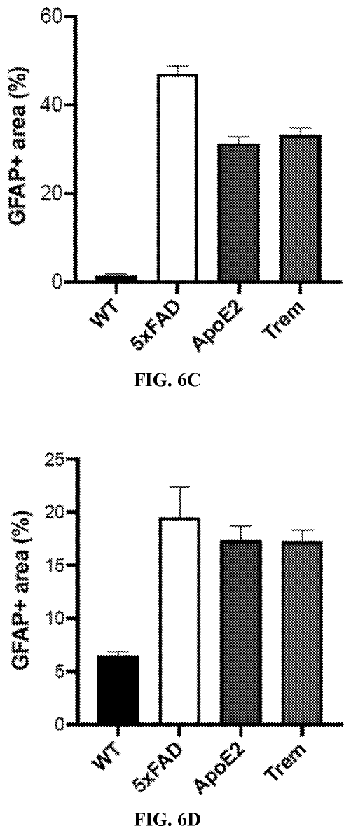

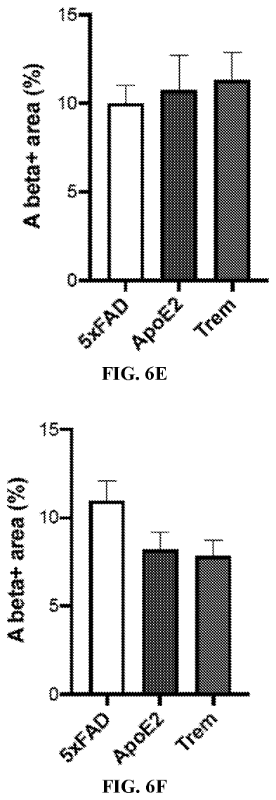

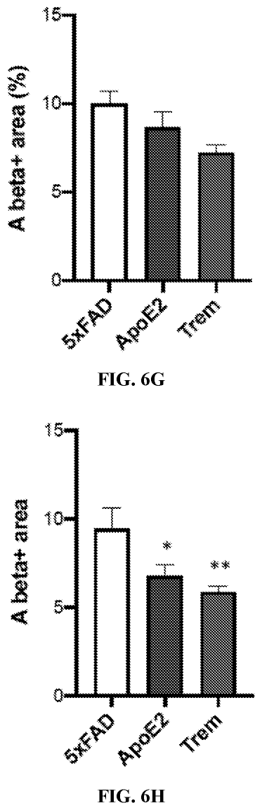

Pathological neuroinflammation is associated with neurodegeneration and primarily mediated by microglia, the resident immune cells of the central nervous system. Microglia has critical functions in sensing environmental changes, responding to harmful stimuli, and phagocytosing debris and apoptotic neurons. Neuroinflammation was assessed by using specific markers for microglial activation (Iba1), and astrocytic response (GFAP) (FIGS. 6A-6D). As expected, the expression of Iba1 and GFAP was higher in 5×FAD compared to the wild type animals. The expression level of these markers was very heterogeneous in treated mice, likely due to differences in the engraftment of transduced cells/their progeny. In particular, Iba1 and GFAP signal in the brain (cortex and hippocampus) of treated mice was reduced of 4 to 70% as compared to the signal measured in control 5×FAD controls; however, significance in the comparison of the entire cohorts was only achieved in the case of Iba1 signal in the cortex region. Amyloid β (Aβ) aggregates trigger a cascade of reactions, involving neuronal cell death, neuroinflammation and gliosis and, ultimately, cognitive impairment. The histological analysis of control 5×FAD brains revealed the intracellular accumulation of plaques containing Aβ deposits (FIGS. 6E-6H). Interestingly, treated animals showed significant reduction in Aβ level in the cortex and hippocampus with dose-dependency: higher rates of Amyloid β (Aβ) reduction were observed in animals showing a vector copy number (VCN) in the brain tissue >0.5, average value of the study cohorts. These effects on neuroinflammation and Amyloid f3 (Aβ) deposition were more pronounced in Trem2-versus ApoE2-treated animals, and were not influenced by the use of the MT1G vector in addition to the ones encoding Trem2 or APoE2.

A hematopoietic stem cell (HSC) gene therapy strategy for Alzheimer's disease (AD) was tested in the most studied disease animal model (5×FAD mice). The gene therapy strategy was based on lentiviral vector (LV)-mediated hematopoietic stem cell (HSC) engineering to express in microglia-like tissue progeny i) Trem2 or ii) ApoE2. In both settings potential synergic effects of MT1G expression with the two therapeutic constructs was tested.

New lentiviral vectors were developed for the expression of potential therapeutic transcripts in Alzheimer's disease (AD) central nervous system (CNS) for impacting key molecular mechanisms of disease. In particular, Trem2 and ApoE2 were expressed. The vectors were produced in the form of vesicular stomatitis virus G (VSV-G) pseudotyped lentival vectors (LV) for the transduction of microglia cell lines and hematopoietic stem progenitor cells (HSPCs). In vitro transduction of BV-2 cells with Trem2 encoding lentiviral vectors (LVs) demonstrated an enhancement of Amyloid β (Aβ) oligomers engulfment, supporting the choice of the transgene.

These vectors were used for an in vivo intra-CNS (central nervous system) hematopoietic stem cell (HSC) gene therapy study in 5×FAD mice intended at assessing whether any of these two strategies had any impact on disease progression. 5×FAD hematopoietic stem cells (HSCs) transduced with the two therapeutic lentiviral vectors (LVs), engrafted in the brain of intra-cerebral ventricular (ICV) transplanted, myeloablated 5×FAD mice. Behavioral and histological analyses of these mice at comparison to mock treated controls indicated that the constructs, especially the Trem2, likely has a positive impact on the onset and early stages of the disease. Ameliorated behavior, reduction of neuroinflammation and a LV-dose dependent reduction of Amyloid β (Aβ) deposits were observed. For all the evaluations, some variability is present related to technical issue that accounted for generally lower-than-targeted transduced cell engraftment in the central nervous system (CNS) in all the cohorts.

Trem2-treated animals showed promising results as treatment attenuated disease clinical manifestation, as per behavioral studies, and disease-associated histological abnormalities.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. This application may be related to PCT/US2020/045106 and PCT/US2017/05677, as well as the PCT applications, entitled “MICROGLIA SPECIFIC PROMOTERS AND METHODS OF USE THEREFORE” and “COMPOSITIONS AND METHODS FOR TREATING AMYOTROPHIC LATERAL SCLEROSIS”, each filed Oct. 1, 2020, which claim priority to the following provisional applications, respectively, 62/908,966 and 62/908,942, all of which are incorporated herein by reference.

1. A method of treating a subject having or having a propensity to develop Alzheimer's disease, the method comprising administering to the subject an effective amount of a cell comprising an expression vector or expression cassette comprising one or more polynucleotides encoding an ApoE2 polypeptide and a Trem2 polypeptide.

2. An expression vector or expression cassette comprising a polynucleotide encoding the following polypeptides: ApoE2 and metallothionein 1G, TREM2 and metallothionein 1G, ApoE2 and TREM2, or ApoE2, TREM2 and metallothionein 1G (MT1G).

3. (canceled)

4. The method of claim 1, wherein the vector or cassette comprises two or more copies of a metallothionein.

5. The method of claim 1, wherein the vector comprises a polynucleotide encoding at least four copies of MT1G.

6. The method of claim 1, wherein the vector comprises a promoter driving expression of the polynucleotide.

7. The method of claim 6 or the expression vector or expression cassette of claim 6, wherein the promoter is the human phosphoglycerate kinase promoter or a microglia specific promoter.

8. (canceled)

9. The method of claim 7, wherein the promoter is a TSPO, MHC class II, or CX3CR1 promoter.

10. (canceled)

11. The method of claim 1, wherein the vector is a lentiviral vector.

12. A lentiviral vector comprising a phosphoglycerate kinase (PGK) promoter or microglia specific promoter driving the expression of a polynucleotide encoding a TREM2 polypeptide and an ApoE2 polypeptide, or fragments thereof and further comprising one or more copies of a polynucleotide encoding metallothionein.

13-14. (canceled)

15. A cell comprising the vector of claim 2.

16. The cell of claim 15, wherein the cassette is inserted at a CX3CR1 or a TSPO gene locus.

17. The cell of claim 15, wherein the cell is a microglial cell or a precursor thereof, a hematopoietic stem cell, a hematopoietic stem progenitor cell (HSPC) or a cell descended from the hematopoietic stem cell or a hematopoietic stem progenitor cell.

18. The cell of claim 17, wherein the HSPC is CD34⁺ and/or CD38⁻ and/or CD90⁺.

19. The cell of claim 15, wherein the cell is hemizygous for the CX3CR1 gene.

20. A method of reducing amyloid beta levels in a cell or tissue or increasing engulfment of beta amyloid by a cell, the method comprising contacting the cell with a polynucleotide encoding two or more of an ApoE2 polypeptide, a Trem2 polypeptide, and a metallothionein polypeptide or fragments thereof.

21-24. (canceled)

25. The method of claim 20, wherein the polynucleotide encodes ApoE2, and TREM2, ApoE2 and metallothionein 1G, TREM2 and metallothionein 1G, or ApoE2, TREM2 and metallothionein 1G (MT1G).

26-33. (canceled)

34. A method of treating a subject having or having a propensity to develop Alzheimer's disease or neuroinflammation, the method comprising administering to the subject an effective amount of a cell comprising a polynucleotide encoding two or more of an ApoE2 polypeptide, and a Trem2 polypeptide, and a metallothionein polypeptide, or fragments thereof.

35-38. (canceled)

39. The method of claim 34, wherein the cell is administered intracerebroventricularly, intravenously, or intrathecally.

40-63. (canceled)

64. A pharmaceutical composition comprising the HSPC of claim 17.

65. A kit comprising the HSPC of claim 17 and directions for its delivery to a subject.
